(19) United States
(12) Patent Application Publication
(10) Pub. No.: US 2016/0228521 A1
(43) Pub. Date: Aug. 11, 2016

(54) MANAGING IMMUNE RESPONSES IN TRANSPLANTATION

(71) Applicant: ALLERTEIN THERAPEUTICS, LLC, Fairfield, CT (US)

(72) Inventors: Howard SOSIN, Fairfield, CT (US); Michael CAPLAN, Fairfield, CT (US)

(73) Assignee: ALLERTEIN THERAPEUTICS, LLC, Fairfield, CT (US)

(21) Appl. No.: 15/022,052

(22) PCT Filed: Sep. 15, 2014
(86) PCT No.: PCT/US14/55625

§ 371 Date: Mar. 15, 2016

Related U.S. Application Data
(60) Provisional application No. 61/878,525, filed on Sep. 16, 2013.

(51) Int. Cl. A61K 39/00; A61K 9/50; A61K 9/51

(52) U.S. Cl. CPC A61K 39/001; A61K 9/51; A61K 9/5068; A61K 2039/542; A61K 2039/577; A61K 2039/55

(57) ABSTRACT

The present invention provides, among other things, methods of treatment of donor and/or host subjects prior to, during, and/or after transplantation of donor tissue into the host subject with nanoparticle compositions encapsulating tissue components to usefully manage immune responses associated with transplantation. In some embodiments, methods include administering to a recipient organism who has received or will receive a transplant of one or more heterologous tissue components from a donor organism a composition including encapsulated donor organism tissue components. In some embodiments, methods include administering to a donor organism from which one or more donor tissue components are to be transplanted into a recipient organism a composition including one or more encapsulated recipient tissue components. In some embodiments, methods include administering to the recipient organism into which one or more donor tissue components are to be transplanted from the donor organism one or more encapsulated MHC proteins.

### BACKGROUND

Tissue transplantation technologies are powerful tools capable of restoring compromised or lost functions to an organism. From cardiac transplantation to corneal grafts, tissue transplantation has been applied in a wide variety of medical contexts, often with beneficial results. However, undesirable immune reactions, including host rejecting grafted tissue and/or grafted tissue attacking host tissues, can disrupt or even destroy the effectiveness or utility of these important technologies.

### SUMMARY

The present invention encompasses the recognition that treatment of donor and/or host subjects prior to, during, and/or after transplantation of donor tissue into the host subject with encapsulated tissue components can usefully manage immune responses associated with transplantation. For example, in some embodiments, the present invention provides the insight that certain undesirable immune reactions associated with tissue transplant (i.e., introduction of tissue components into a recipient organism from an external source) can be treated (e.g., delayed, suppressed, reduced [e.g., in frequency, severity, and/or intensity], and/or eliminated) by administration of appropriate tissue component compositions as described herein to the recipient organism prior to, during, and/or after the transplant. Alternatively or additionally, in some embodiments, the present invention provides the insight that certain undesirable immune reactions associated with such tissue transplant can be treated by administration of appropriate tissue component compositions as described herein to a donor organism from which transplanted tissue components are obtained or derived, prior to, during, and/or after such tissue components (or their precursors) are obtained from the donor organism.

The present invention provides, among other things, tissue component compositions, methods for administering provided tissue component compositions, and methods of forming provided tissue component compositions.

In some embodiments, tissue components are encapsulated within one or more nanoparticles to form tissue component compositions. In some embodiments, nanoparticles within provided tissue component compositions are comprised of polymers, polymeric entities, and/or amphiphilic entities.

In some embodiments, tissue components within provided tissue component compositions are encapsulated in one or more cells. In some embodiments, cells are comprised of living cells; in some embodiments, cells are comprised of dead (e.g., killed) cells. In some embodiments, cells are comprised of microbial cells.

In some embodiments, nanoparticles and/or cells encapsulate tissue components. In some embodiments, nanoparticles and/or cells encapsulate one or more heterologous tissue components. In some embodiments, nanoparticles and/or cells encapsulate one or more recipient tissue components. In some embodiments, nanoparticles and/or cells encapsulate one or more MHC proteins.

In some embodiments, the present invention provides tissue component compositions formulated for intravenous, intradermal, transdermal, oral, subcutaneous, and/or transmucosal administration.

Still further, the present invention provides tissue component compositions formulated for transmucosal delivery via buccal, nasal, bronchial, vaginal, rectal, and/or sublingual administration.

The present invention provides, among other things, methods including steps of administering to a recipient organism who has received or will receive a transplant of one or more heterologous tissue components from a donor organism a composition comprising encapsulated donor organism tissue components.

Alternatively or additionally, in some embodiments, the present invention provides methods including steps of administering to a donor organism from which one or more donor tissue components are to be transplanted into a recipient organism a composition comprising one or more encapsulated recipient tissue components.

The present invention also provides, among other things, methods including steps of determining which MHC proteins are expressed by a donor organism, determining which MHC proteins are expressed by a recipient organism, selecting one or more encapsulated MHC proteins that matches one or more MHC proteins of the donor organism, and administering to the recipient organism into which one or more donor tissue components are to be transplanted from the donor organism the one or more encapsulated MHC proteins.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The Figures described below, that together make up the Drawing, are for illustration purposes only, not for limitation.

FIG. 1: depicts an exemplary schematic, according to some embodiments, of a nanoparticle encapsulating donor tissue and/or donor MHC molecules. FIG. 1A depicts an exemplary schematic, according to some embodiments, of a nanoparticle encapsulating donor tissue (“DT”). FIG. 1B depicts an exemplary schematic, according to some embodiments, of a nanoparticle encapsulating donor MHC molecules (“MHC”). In some embodiments, LPS and other bacterial components (“other”) are used to coat the outer surface of the nanoparticles. Without wishing to be bound to a particular theory, in some embodiments these bacterial components facilitate uptake by APCs, thereby increasing drug potency.

FIG. 2: depicts an exemplary transplant timeline of nanoparticle administration. In some embodiments, primary administration is followed by one or more secondary administrations (e.g., 2, 3, 4, 5, 6, 7, 8, 9, 10, 15, 20, or more) together which reduce, modulate, and/or eliminate host immune response to the transplanted tissue(s), after which tissue is transplanted into the host. Alternatively or additionally, in some embodiments, nanoparticles are administered in one or more optional post-graft administrations (e.g., 2, 3, 4, 5, 6, 7, 8, 9, 10, 15, 20, or more).

FIG. 3: depicts a flowchart of an exemplary xenograft (pig donor to human host) tissue transplant of a porcine heart valve. Donor porcine tissue components are encapsulated within nanoparticles, which are then administered to the host. In some embodiments, donor porcine tissue components are administered to the host two or more times (e.g., 3, 4, 5, 6, 7, 8, 9, 10, 15, 20, or more) prior to the actual transplant.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; (iv) the terms “about” and “approximately” may be understood to permit standard variation as would be understood by those of ordinary skill in the art; and (v) where ranges are provided, endpoints are included.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Amino acid: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

Animal: As used herein, the term “animal” refers to any member of the animal kingdom. In some embodiments, “animal” refers to humans, at any stage of development. In some embodiments, “animal” refers to non-human animals, at any stage of development. In some embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Antigen presenting cell: The phrase “antigen presenting cell” or “APC,” as used herein, has its art understood meaning referring to cells which process and present antigens to T-cells. Exemplary antigen presenting cells include dendritic cells, macrophages and certain activated epithelial cells.

Approximately: As used herein, the term “approximately” and “about” is intended to encompass normal statistical variation as would be understood by those of ordinary skill in the art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are “associated” with one another if they interact, directly or indirectly, so that they are and remain in physical proximity with one another.

Autoantigen: As used herein, the term “autoantigen” is used to refer to antigens produced by an individual that are recognized by the immune system of that individual. In some embodiments, an autoantigen is one whose recognition by the individual's immune system is associated with an autoimmune disease, disorder or condition. In general, an autoantigen may be or include any chemical entity such as, for example, a small molecule, a nucleic acid, a polypeptide, a carbohydrate, a lipid, etc. In some embodiments, an autoantigen is or comprises a polypeptide. Those of skill in the art are familiar with a variety of agents, including polypeptides, that can act as autoantigens, and particular that are recognized in immune reactions associated with autoimmunity diseases, disorders and/or conditions.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Biologically active: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a lysate of one or more cells or tissues from a transplant donor; in some embodiments a cellular lysate is a lysate of one or more cells or tissues from an intended or actual transplant recipient or host. In some embodiments, a cellular lysate is a crude lysate in that little or no purification is performed after disruption of the cells, which generates a “primary” lysate. In some embodiments, one or more isolation or purification steps is performed on the primary lysate. However, the term “lysate” refers to a preparation that includes multiple cellular components and not to pure preparations of any individual component.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Combination therapy: As used herein, the term “combination therapy” refers to those situations in which a subject is simultaneously exposed to two or more therapeutic agents. In some embodiments, such agents are administered simultaneously; in some embodiments, such agents are administered sequentially; in some embodiments, such agents are administered in overlapping regimens.

Corresponding to: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190^{th} residue in the first polymer but rather corresponds to the residue found at the 190^{th} position in the reference polymer; those of ordinary skill in the art readily appreciate how to identify “corresponding” amino acids, including through use of one or more commercially-available algorithms specifically designed for polymer sequence comparisons.

Derivative: As used herein, the term “derivative” refers to a structural analogue of a reference substance. In some embodiments, a derivative is produced or formed from, and/or designed with reference to, the reference substance. In some embodiments, a derivative refers to a second chemical substance related structurally to a first chemical substance and theoretically derivable from the first chemical substance.

Donor tissue: As used herein, the phrase “donor tissue” refers to tissue or tissue components (e.g., cells or components thereof) implanted or prepared for implantation into a host/recipient. In some embodiments, donor tissue is obtained or derived from a source of a different species from the host/recipient. In some embodiments, donor tissue is obtained or derived from a source of the same species as the host/recipient. In some embodiments, donor tissue is obtained or derived from the host/recipient.

Dosage form: As used herein, the term “dosage form” refers to a physically discrete unit of a therapeutic agent for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is correlated with a desired or beneficial outcome when administered across a relevant population (i.e., is a therapeutic dosing regimen).

Encapsulated: The term “encapsulated” is used herein to refer to substances that are completely surrounded by another material.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Functional: As used herein, the term “functional” is used to refer to a form or fragment of an entity that exhibits a particular property and/or activity.

Graft rejection: The term “graft rejection” as used herein, refers to rejection of tissue transplanted from a donor individual to a recipient individual. In some embodiments, graft rejection refers to an allograft rejection, wherein the donor individual and recipient individual are of the same species. Typically, allograft rejection occurs when the donor tissue carries an alloantigen against which the recipient immune system mounts a rejection response. In some embodiments, graft rejection refers to a xenograft rejection, wherein the donor and recipient are of different species. Typically, xenograft rejection occurs when the donor species tissue carries a xenoantigen against which the recipient species immune system mounts a rejection response.

Heterologous tissue: The phrase “heterologous tissue”, as used herein, refers to tissue from an immunologically distinct source (e.g., a non-self source). In some embodiments, heterologous tissue is tissue from a first organism of a first species that is to be transplanted into a second organism, of the same or a different species.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Host: The term “host” is used herein to refer to an organism that receives a transplant. In some embodiments, the host is of a different species than is the source of the donor tissue (e.g., than is the donor or the source from which cells of the donor tissue were derived). In some embodiments, the host is of the same species as is the source of the donor. In some embodiments, the host is the source of the donor tissue.

Human: In some embodiments, a human is an embryo, a fetus, an infant, a child, a teenager, an adult, or a senior citizen.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent identity between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. Representative algorithms and computer programs useful in determining the percent identity between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent identity between two nucleotide sequences can, alternatively, be determined for example using the GAP program in the GCG software package using an NWSgapdna.CMP matrix.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients.

Microorganisms: As used herein, the term “microorganisms” refers to cells, bacteria, fungi, Archaea, and/or protozoa. Preferred microorganisms can be genetically manipulated to produce a desired polypeptide(s).

Nanoparticle: The term “nanoparticle” is used herein to refer to a macromolecular entity whose surface forms a boundary with a medium in which the nanoparticle is present. In some embodiments, a nanoparticle has an internal lumen (e.g., is a micelle or has micellular structure); in some such embodiments, the lumen is bounded by a membrane. In some embodiments, the membrane has an inner surface facing the lumen. In some embodiments, a nanoparticle comprises a membrane with an outer surface facing the external medium in which the nanoparticle is present. In some embodiments, a nanoparticle is substantially solid in that it does not have a clearly distinct lumen. In some embodiments, a nanoparticle or nanoparticle membrane is comprised of a polymer, a polymeric entity, and amphiphilic entity (e.g., a lipid), or combinations thereof. Typically, a nanoparticle has a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health.

Nanoparticle composition: As used herein, the term “nanoparticle composition” refers to a composition that contains at least one nanoparticle and at least one additional agent or ingredient. In some embodiments, a nanoparticle composition contains a substantially uniform collection of nanoparticles as described herein.

Nanoparticle membrane: As used herein, the term “nanoparticle membrane” refers to the boundary or interface between a nanoparticle outer surface and a surrounding environment. In some embodiments, the nanoparticle membrane has an outer surface and bounds a lumen. In some embodiments, a nanoparticle membrane is comprised of one or more polymers, polymeric components, and/or amphiphilic entities. In some embodiments, a nanoparticle membrane has a single layer. In some embodiments, a nanoparticle membrane has a plurality of layers (e.g., is a bilayer, trilayer, tetralayer, etc.). In some embodiments, one or more entities or agents (e.g., polypeptide agents such as, for example, glycopeptide and/or lipopeptide agents) is partially or wholly contained within a nanoparticle membrane; in some such embodiments at least a portion of the entity or agent is exposed on the membrane surface or to the lumen. In some embodiments, an entity or agent at least partially contained in the membrane crosses the membrane at least once. In some embodiments, an entity or agent at least partially contained in the membrane crosses the membrane multiple times. In many embodiments, portions of structure that cross the membrane are referred to as “membrane-spanning” or “trans-membrane” regions or moieties.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Patient: As used herein, the term “patient” or “subject” refers to a human or any non-human animal (e.g., mouse, rat, rabbit, dog, cat, cattle, swine, sheep, horse or primate) to whom therapy is administered. In many embodiments, a patient is a human being. In some embodiments, a patient is a human presenting to a medical provider for diagnosis or treatment of a disease, disorder or condition. In some embodiments, a patient displays one or more symptoms or characteristics of a disease, disorder or condition. In some embodiments, a patient does not display any symptom or characteristic of a disease, disorder, or condition. In some embodiments, a patient is someone with one or more features characteristic of susceptibility to or risk of a disease, disorder, or condition.

Pharmaceutically acceptable: The term “pharmaceutically acceptable” as used herein, refers to agents that, within the scope of sound medical judgment, are suitable for use in contact with tissues of human beings and/or animals without excessive toxicity, irritation, allergic response, or other problem or complication, commensurate with a reasonable benefit/risk ratio.

Polypeptide: The term “polypeptide”, as used herein, generally has its art-recognized meaning of a polymer of at least three amino acids. In some embodiments, a polypeptide is a MHC molecule. In some embodiments, the term is used to refer to specific functional classes of polypeptides, such as, for example, autoantigen polypeptides, nicotinic acetylcholine receptor polypeptides, alloantigen polypeptides, etc. For each such class, the present specification provides several examples of amino acid sequences of known exemplary polypeptides within the class; in some embodiments, such known polypeptides are reference polypeptides for the class. In such embodiments, the term “polypeptide” refers to any member of the class that shows significant sequence homology or identity with a relevant reference polypeptide. In many embodiments, such member also shares significant activity with the reference polypeptide. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region, often including a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Self tissue: The phrase “self tissue” is used herein to refer to tissue of an individual of interest. That is, for any particular individual, “self tissue” is tissue of that individual. In some embodiments, self tissue may be defined as i) tissue that the individual's immune system treats as self; and/or ii) tissue that originated in the individual. In some embodiments, the individual is or will be (e.g., is scheduled to be) a transplant donor; in some embodiments, the individual is or will be (e.g., is scheduled to be) a transplant recipient.

Small molecule: As used herein, the term “small molecule” means a low molecular weight organic compound that may serve as an enzyme substrate or regulator of biological processes. In general, a “small molecule” is a molecule that is less than about 5 kilodaltons (kD) in size. In some embodiments, provided nanoparticles further include one or more small molecules. In some embodiments, the small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, one or more small molecules are encapsulated within the nanoparticle. In some embodiments, small molecules are non-polymeric. In some embodiments, in accordance with the present invention, small molecules are not proteins, polypeptides, oligopeptides, peptides, polynucleotides, oligonucleotides, polysaccharides, glycoproteins, proteoglycans, etc. In some embodiments, a small molecule is a therapeutic. In some embodiments, a small molecule is an adjuvant. In some embodiments, a small molecule is a drug.

Stable: The term “stable,” when applied to compositions herein, means that the compositions maintain one or more aspects of their physical structure (e.g., size range and/or distribution of particles) over a period of time. In some embodiments, a stable nanoparticle composition is one for which the average particle size, the maximum particle size, the range of particle sizes, and/or the distribution of particle sizes (i.e., the percentage of particles above a designated size and/or outside a designated range of sizes) is maintained for a period of time under specified conditions. In some embodiments, a stable provided composition is one for which a biologically relevant activity is maintained for a period of time. In some embodiments, the period of time is at least about one hour; in some embodiments the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. For example, if a population of nanoparticles is subjected to prolonged storage, temperature changes, and/or pH changes, and a majority of the nanoparticles in the composition maintain a diameter within a stated range, the nanoparticle composition is stable. In some embodiments, a stable composition is stable at ambient conditions. In some embodiments, a stable composition is stable under biologic conditions (i.e. 37° C. in phosphate buffered saline).

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition has been diagnosed with and/or exhibits or has exhibited one or more symptoms or characteristics of the disease, disorder, or condition.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, a risk of developing a disease, disorder, and/or condition is a population-based risk (e.g., family members of individuals suffering from allergy, etc.).

Symptoms are reduced: According to the present invention, “symptoms are reduced” when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom.

Therapeutic agent: As used herein, the phrase “therapeutic agent” refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition (e.g., host versus graft disease). In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective agent may be formulated and/or administered in a single dose. In some embodiments, a therapeutically effective agent may be formulated and/or administered in a plurality of doses, for example, as part of a dosing regimen.

Therapeutic regimen: A “therapeutic regimen”, as that term is used herein, refers to a dosing regimen whose administration across a relevant population is correlated with a desired or beneficial therapeutic outcome.

Tissue components: “Tissue components”, as that term is used herein, are intact or disrupted tissue materials. In some embodiments, tissue components comprise intact tissue samples, e.g., obtained from an organism or from an organism's cells. In some embodiments, tissue components comprise cells of a relevant type as found in a tissue of interest. In some embodiments, tissue components comprise cells obtained from a tissue sample. In some embodiments, tissue components comprise some or all contents of one or more cells. In some embodiments, tissue components comprise living cells. In some embodiments, tissue components comprise dead or killed cells. In some embodiments, tissue components comprise disrupted cells. In some embodiments, tissue components comprise cell parts less than complete cells. In some embodiments, tissue components comprise a cellular extract, which may be or comprise a cellular lysate. In some embodiments, tissue components are provided in a crude sample, for example, that has been subject to little or no processing beyond separation from its source (e.g., as a primary tissue explant, cell sample, etc.). In some embodiments, tissue components are provided in a purified or processed sample, for example, that was subjected to one or more isolation, purification, and/or processing steps. In some embodiments, tissue components are or comprise substantially pure or purified entities. In some embodiments, tissue components are or comprise one or more entities that has/ve been artificially produced in a system other than a natural organism. In some embodiments, tissue components comprise or consist of MHC molecules, which in some embodiments may be complexed with peptide(s) found in the tissue. In some such embodiments, such complexes are arranged, constructed, and/or assembled so that the peptide(s) is/are presented to relevant immune cells (e.g., T cells, B cells).

Tissue transplantation: The term “tissue transplantation”, as used herein, refers to a transfer of tissue or tissue components from an external source into a recipient (host) individual. In some embodiments, the external source is an organism (e.g., a living, brain dead, recently dead, or dead organism). In some embodiments, the external source is an ex vivo or in vitro system.

Treatment: As used herein, the term “treatment” (also “treat” or “treating”) refers to any administration of a substance that partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Uniform: The term “uniform,” when used herein in reference to a nanoparticle composition, refers to a nanoparticle composition in which individual nanoparticles have diameters within a specified range. For example, in some embodiments, a uniform nanoparticle composition is one in which the difference between the minimum diameter and maximum diameter does not exceed about 300 nm. In some embodiments, a uniform nanoparticle composition contains nanoparticles with diameters within the range of about 100 nm to about 300 nm. In some embodiments, a uniform nanoparticle composition contains nanoparticles with an average particle size that is under about 500 nm. In some embodiments, a uniform nanoparticle composition contains nanoparticles with an average particle size that is within a range of about 100 nm to about 500 nm. In some embodiments, a uniform nanoparticle composition is one in which a majority of the particles within the composition have diameters below a specified size or within a specified range. In some embodiments, the majority is more than 50%, 60%, 70%, 75%, 80%, 85%, 90%, 95%, 96%, 97%, 98%, 99%, 99.5%, 99.6%, 99.7%, 99.8%, 99.9% or more of the particles in the composition.

### DETAILED DESCRIPTION OF CERTAIN EMBODIMENTS

Among other things, the present invention provides the insight that certain undesirable immune reactions associated with tissue transplant (i.e., introduction of tissue components into a recipient organism from an external source) can be treated (e.g., delayed, suppressed, reduced [e.g., in frequency, severity, and/or intensity], and/or eliminated) by administration of appropriate tissue component compositions as described herein to the recipient organism prior to during, and/or after the transplant. Alternatively or additionally, in some embodiments, the present invention provides the insight that certain undesirable immune reactions associated with such tissue transplant (e.g. graft-versus host disease) can be treated by administration of appropriate tissue component compositions as described herein to a donor organism from which transplanted tissue components are obtained or derived, prior to, during, and/or after such tissue components (or their precursors) are obtained from the donor organism.

In some embodiments, the present invention encompasses the recognition that treatment of a recipient organism with tissue component compositions as described herein that include tissue components from the external source of tissue that is or will be transplanted into the recipient (e.g., donor tissue components) can treat (e.g., delay, suppress, reduce [e.g., in frequency, severity, and/or intensity] and/or eliminate undesirable immune reactions associated with tissue transplant.

In some embodiments, the present invention encompasses the recognition that treatment of a donor organism from which donor tissue components (or their precursors) is or will be obtained or derived can treat (e.g., delay, suppress, reduce [e.g., in frequency, severity, and/or intensity] and/or eliminate undesirable immune reactions associated with tissue transplant.

### Major Histocompatibility Complex and Immune System Activation

The major histocompatibility complex (MHC) gene family is a series of genes that code for MHC protein molecules responsible for cell-cell recognition and interaction. The MHC gene family of mammalian species contains three groups of genes: class I, class II, and class III. Class I and class II genes code for cell surface recognition molecules. Class III genes code for certain complement components. In humans, MHC molecules are also known as human leukocyte antigens (HLAs).

The ability of cells to recognize other cells as self or as originating from another genetically different individual (non-self) is an important property in maintaining the integrity of tissue and organ structure. Class I and class II MHC products control recognition of self and non-self. The major histocompatibility system thus prevents an individual from being invaded by cells from another individual. For example, transplants from one individual generally cannot survive in another individual because of histocompatibility differences.

Histocompatibility similarities are required for cellular cooperation in induction of the immune response, and they provide a mechanism to ensure that T cells and B cells of a given individual can recognize each other for cooperation, yet recognize foreign structures at the same time. For instance, T lymphocytes, when presented with an antigen in the proper manner, react in one of three ways: through the generation of T cytotoxic lymphocytes (T_{C}), through amplification by T helper cells (T_{H}), or through suppression by T suppressor cells (T_{S}) of the effects of other T or B cells. In general, T lymphocytes only recognize the antigen and respond to it when it is presented on the surface of antigen-presenting cell. This antigen-presenting cell may vary according to the type of T lymphocyte involved. Thus, in the generation of cytotoxic responses, lymphocytes and possibly macrophages present the antigen to the T_{C} cells, while in the other types of T response the presenting cell may be a macrophage and perhaps dendritic cells.

In general, T cells need to recognize two structures, a foreign antigen and an MHC gene product, for their subsequent activation. The process of generating T_{C} cells and a cytotoxic response requires that the antigen be presented to the T cells in association with an MHC class I gene product. On the other hand, for B cells to be activated, binding to the antigen is necessary, plus a second signal usually given by a T_{H} lymphocyte. However, the T_{H} lymphocytes require the presentation of the antigen in a processed form by an antigen-presenting cell in the context of an MHC class II determinant. Exemplary MHC-T Cell Receptor interactions may be found in Hennecke et al., T Cell Receptor-MHC Interactions Up Close, 2001, Cell, 104:1-4, the disclosure of which is hereby incorporated by reference.

In the case of B cell activation, whatever the antigen-presenting cell is, it must process the antigen before presenting it to the T_{H} lymphocytes. This involves taking up the antigen, sequestering it in intracellular compartments, and re-expressing the antigen or a portion thereof on the surface of the antigen-presenting cell in association with a class II MHC determinant. The T_{H} cell must be able to recognize the processed antigen and class II markers on both the antigen-presenting cell and the B cell. When each of these requirements is fulfilled, the B cell will be stimulated to proliferate, which greatly increases the number of cells capable of synthesizing specific antibody. These then differentiate into plasma cells, which secrete large amounts of antibody. A similar response employing class II receptors on T_{S} suppressor cells and class II MHC markers on macrophages and B cells may be operative in induction of T suppressor activity, which turns off antibody production.

The MHC is located on chromosome 6 in humans and extends over 4 million base pairs, containing more than 200 genes. There are three class I α-chain genes in humans: HLA-A, -B, and -C. There are also three pairs of MHC Class II α- and β-chain genes, called HLA-DR, -DP, and -DQ. The MHC genes are also highly polymorphic, allowing for a wide array of potential MHC molecules. In addition to the disclosure presented below, more information regarding MHC structure and the structure of MHC-peptide interactions as well as antigen presentation by MHC Class I and II molecules may be found, inter alia, in Madden D R, The Three Dimensional Structure of Peptide-MHC Complexes, Ann. Rev. Immunol., 1995, 13:587-622; and Neefjes et al., Towards a Systems Understanding of MHC Class I and MHC Class II Antigen Presentation, 2011, Nat. Rev. Immunol., 11(12): 823-836, the disclosures of which are hereby incorporated by reference in their entirety.

Class I MHC molecules are composed of two polypeptide chains, a long α chain and a short β chain called β2-microglobulin. The α chain of MHC Class I molecules includes four regions: a cytoplasmic region, a transmembrane region containing hydrophobic amino acids by which the molecule is anchored in the cell membrane, a highly conserved α3 immunoglobulin-like domain which binds to CD8, and a highly polymorphic peptide binding region that forms from the α1 and α2 domains. The peptide binding domain exhibits the highest variability in the MHC Class I molecule and contains a groove that will accommodate peptides of approximately 8-10 amino acids in length, or portions of a longer polypeptide. The binding of a peptide to the peptide binding groove is an essential step in the synthesis of MHC Class I molecules and it is this binding that stabilizes the molecule and allows translocation form the endoplasmic reticulum to the cell membrane. Each peptide binding region may bind a variety of peptides, for example, those from an infectious pathogen or other undesirable foreign peptide However, it is of note that, even in the absence of infection, there is typically a steady supply of peptides from the cytosol into the ER which may bind to the peptide binding region of the MHC Class I molecule. Exemplary non-foreign proteins that may bind to the peptide binding region include defective ribosomal products and old or non-functional proteins marked for destruction.

Class II MHC molecules are composed of two polypeptide chains, an α and a β chain of approximately equal length. Both chains include four regions: a cytoplasmic region containing sites for phosphorylation and binding to cytoskeletal elements, a transmembrane region containing hydrophobic amino acids by which the molecule is anchored in the cell membrane, a highly conserved α2 domain and a highly conserved β2 domain to which CD4 may bind, and a highly polymorphic peptide binding region formed form the α1 and β1 domains. In many ways, the peptide binding region is very similar to that of Class I MHC molecules. However, the groove formed in the peptide binding region of Class II MHC molecules is larger and may accommodate peptides between 13-25 amino acids in length, or portions of larger peptides.

### Tissue Transplantation

For dysfunctional, diseased, and/or otherwise undesired tissues or organs of the body, besides therapeutic intervention with drugs, organ and/or tissue transplantation is an alternative, and, in some cases, the last resort in the treatment of a patient. Particularly for patients with leukemia, end-stage renal, cardiac, pulmonary or hepatic failure, organ transplantation is quite commonly used in the treatment.

In some embodiments, allografts (organ grafts harvested from donors other than the patient him/herself or host/recipient of the graft) of various types, e.g. kidney, heart, lung, liver, bone marrow, pancreas, cornea, small intestine and skin (e.g. epidermal sheets) may be used. In some embodiments, xenografts (organ grafts harvested from non-human animals), such as porcine heart valves, may be used to replace their dysfunctional human counterparts.

As an example, in some embodiments, tissue transplantation is or comprises bone marrow and/or stem cell transplantation. Bone marrow and/or stem cell transplantation has applications in a wide variety of clinical settings, including solid organ transplantation. A major goal in solid organ transplantation is the engraftment of the donor organ without a graft rejection immune response generated by the recipient, while preserving the immunocompetence of the recipient against other foreign antigens. Typically, to prevent an undesired immune response, nonspecific immunosuppressive agents such as cyclosporin A, azathioprine, corticosteroids including prednisone, and methylprednisolone, cyclophosphamide, and FK506 are used. However, these agents must typically be administered on a daily basis and if stopped, graft rejection usually results. However, nonspecific immunosuppressive agents function by suppressing all aspects of the immune response, thereby greatly increasing a recipient's susceptibility to infections and diseases, including cancer.

As another example, in some embodiments, tissue transplantation is or comprises hematopoietic tissue transplantation (e.g. bone marrow transplantation). In many embodiments, a goal of hematopoietic tissue transplantation is to achieve the successful engraftment of donor cells within a recipient host, such that immune and/or hematopoietic chimerism results. Chimerism is the reconstitution of the various compartments of the recipient's hematoimmune system with donor cell populations bearing MHC molecules derived from both, the allogeneic or xenogeneic donor, and a cell population derived from the recipient or, alternatively, the recipient's hematoimmune system compartments which can be reconstituted with a cell population bearing MHC molecules derived from only the allogeneic or xenogeneic marrow donor. Chimerism may vary from 100% (total replacement by allogenic or xenogeneic cells) to low levels detectable only by molecular methods. Chimerism levels may vary over time and be permanent or temporary.

In some embodiments, to ensure successful transplantation, it may be desirable to obtain the graft from the patient's identical twin or his/her immediate family member to increase MHC histocompatibility (discussed above). As discussed further below, transplants may evoke a variety of immune responses in the host, including, but not limited to, rejection of the graft by the host immune system, known as graft-versus-host disease (hereinafter, referred to as “GvHD”) in which the transplanted immune system cells (e.g. bone marrow or hematopoietic cell transplants) cause an attack of host tissues often leading to severe complications.

As with any medical procedure, some organ and tissue transplants are more successful than others. If rejection of the tissue or organ begins, administration of immunosuppressive drugs may delay, reduce or stop the rejection. However, patients are required to take immunosuppressive drugs for the rest of their lives. Chronic rejection (discussed below) is the leading cause of tissue and organ transplant failure. In addition, transplant recipients may be prone to certain cancers (e.g., in some patients who take strong immune suppressing drugs for a long time), infections (e.g., because immune system is suppressed), side effects of medications, and/or loss of function in the transplanted tissue/organ.

Despite all of the potential complications, tissue transplantation is a desirable and beneficial procedure in many clinical contexts. The benefits of tissue and organ transplant are significant and include, but are not limited to, extension of life and/or improvement of the quality of life of the recipient. Tissues that have been the subject of successful transplant include heart, heart valve, lung, corneal, kidney, stomach, pancreatic, liver, intestine, joint (including whole knee), ovarian, bone marrow, partial or full facial, jaw, limb (including arm and/or leg), and tracheal tissues.

Undesirable Immune Reactions Associated with Tissue Transplantation

The present invention provides compositions and methods useful in the treatment and/or prevention of undesirable immune reactions (e.g., graft rejection) associated with tissue transplantation.

In some embodiments, undesirable immune reactions include, but are not limited to, host-versus-graft disease, graft-versus-host disease, post-transplant lymphoproliferative disorder (PTLD), bacterial infections, fungal infections, viral infections, gastrointestinal and hepatic complications, neurologic complications, pulmonary complications, and/or combinations thereof.

Host V Graft Disease (HvGD)

Host-versus-graft disease (i.e., transplant rejection; graft rejection, HvGD) occurs when transplanted donor tissue (e.g., tissue transplantation, implantation, graft, etc.) is rejected by the host's (i.e., recipient's) immune system, which attacks the transplanted donor tissue due to differences in human leukocyte antigen haplotypes between the donor and recipient. Essentially, the infection-fighting system of the host recognizes the infused donor cells as being different and works to destroy them. The severity and/or type of transplant rejection may vary and includes hyperacute rejection, acute rejection, chronic rejection; and/or combinations thereof.

Hyperacute rejection is initiated by preexisting humoral immunity and typically occurs when donor/host antigens are not matched. Hyperacute rejection manifests severely within minutes after a recipient receives a transplant, and the only treatment is immediate removal of the transplanted tissue. Hyperacute rejection may also result in systemic inflammatory response syndrome (SIRS). SIRS is a serious condition related to systemic inflammation, organ dysfunction, and organ failure in which there is abnormal regulation of various cytokines (SIRS is a subset of a cytokine storm).

Acute rejection may occur any time from the first week after transplant to three or more months afterwards and occurs to some degree in all transplants unless immunosuppression is achieved. In some embodiments, acute rejection may be treated with immunosuppressive therapy, antibody-based therapy, and/or bone marrow transplant.

Single episodes of acute rejection can be recognized and treated with immunosuppression drugs and/or antibody-based treatments, usually preventing tissue and/or organ failure. However, recurrent acute rejection episodes lead to chronic rejection.

In some embodiments, acute rejection may be treated with one or more bone marrow transplants from the same source as the originally transplanted tissue and/or organ. Hematopoietic stem cells of the bone marrow give rise to all blood cells, including white blood cells which form the immune system. A bone marrow transplant replaces the transplant recipient's immune system with donor bone marrow (e.g., hematopoietic stems cells) from the same transplant source, and as a result the recipient accepts the new tissue and/or organ without rejection. However, bone marrow transplant presents risk of graft versus host disease (GvHD; discussed below in more detail), whereby mature donor lymphocytes entering with donor marrow recognize recipient tissues as foreign and destroy them.

A variety of methods for diagnosing acute rejection are well known in the art and include employing clinical data (i.e., patient signs and symptoms) as well as pathology data (e.g., tissue biopsy). For example, in some embodiments, histological analysis of a tissue biopsy may reveal infiltrating T-cells, structural compromise of tissue anatomy (varying by tissue type transplanted), injury to blood vessels, and/or combinations thereof. In some embodiments, infiltrating T-cells are accompanied by eosinophils, plasma cells, and neutrophils, particularly in telltale ratios.

Chronic rejection takes place over years where the recipient's constant immune response against the new tissue and/or organ(s) slowly damages the transplanted tissues or organ. Typically, chronic rejection manifests as fibrosis (i.e. scarring) of the donated tissue's blood vessels. This type of rejection is often observed in lung transplants, leading to progressive airflow obstruction and, eventually pulmonary insufficiency or one or more secondary acute infections.

Generally, recipient rejection of donor tissue is an adaptive immune response via cellular immunity (mediated by killer T cells inducing apoptosis of target cells) as well as humoral immunity (mediated by activated B cells secreting antibody molecules), though the action is joined by components of innate immune response (phagocytes and soluble immune proteins). In some embodiments, different types of transplanted tissues elicit different balances of rejection mechanisms. Host immunologic mechanisms of rejection include, but are not limited to, immunization, immune system memory, cellular immunity, humoral immunity, and/or combinations thereof.

In some embodiments, a host immunological mechanism of rejection comprises immunization. Immunization occurs when a host is exposed to non-self antigens. In some embodiments, a host's exposure to the antigens of a different member of the same or similar species is referred to as allostimulation, and the tissue is referred to as allogenic tissue. For example, in some embodiments, allogenic donor tissues (e.g. organs) are acquired from a cadaver (e.g. a donor who had succumbed to trauma), whose tissues had already sustained inflammation or ischemia. Antigen presenting cells (e.g. dendritic cells) of the donor tissue migrate to the recipient's peripheral lymphoid tissue, and present the donor's self peptides to the host's immune cells (i.e. lymphocytes). The host's immune cells (i.e. lymphocytes) coordinate specific immunity directed towards the donor's self peptides and/or the donor's MHC molecules.

In some embodiments, an adaptive immune response comprises immune system memory of the host. For example, CD4 receptors of host memory T cells bind MHC class II molecules expressed on the surfaces of select cells; once bound, host memory helper T cell's T cell receptors (TCRs) recognize their target antigen being presented within the MHC class II. Memory helper T cells produce clones that, as effector cells, secrete immune signaling molecules (i.e. cytokines) in approximately the cytokine balance that had prevailed at the memory helper T cell's priming to “memorize” the antigen.

In some embodiments, gastric or hepatic (i.e., liver) diseases are frequent complications that occur after stem cell/bone marrow transplant. Conditioning regimens that usually consist of high-dose chemotherapy, radiation therapy, or both, can cause mucositis. In some embodiments, mucositis is the presence of sores throughout the gastrointestinal tract; signs and symptoms include mouth sores, esophagitis (i.e., soreness when swallowing), stomach ulcers, and/or diarrhea with stomach cramps. In some embodiments, treatment includes intravenous narcotic medications and total parenteral nutrition (TPN) until the mucositis has resolved.

In some embodiments, common hepatic complications that occurs after stem cell transplantation include veno-occlusive disease (VOD) of the liver. Hosts with prior liver injury, a history of hepatitis or a high-risk disorder are at greatest risk of VOD, although the disease can develop in any host after transplantation. In some embodiments, VOD is characterized by elevated concentration of bilirubin which results in the yellow appearance of the skin and eyes, an enlarged liver, fluid retention or weight gain and/or combinations thereof. In some embodiments, VOD is treated by fluid restriction. In some embodiments, preventive measures include the administration of heparin and daily monitoring of weights and fluid balance while the host is hospitalized. VOD can be severe and, in such instances, can even result in death.

In some embodiments, pulmonary or lung complications are significant causes of morbidity and mortality after tissue transplant. For example, in some embodiments, complications include, but are not limited to infectious and non-infectious causes of pneumonitis (i.e., lung inflammation). In some embodiments, pathogens that cause lung infections include bacterial, fungal and viral organisms.

Treatments and Therapies

In some embodiments, to prevent graft rejection, the host receives medications, chemotherapy (i.e., destroy host bone marrow), total body irradiation, and other antibody medications before receiving donor tissue transplant. In some embodiments, chance of graft rejection are related to the match between the donor and host MHC antigens, the overall genetic relationship between donor and host, and the type of disease for which the transplantation has been performed.

In some embodiments, immunosuppressive therapy includes administration of corticosteroids (e.g., prednisolone, hydrocortisone, etc.), calcineurin inhibitors (e.g., cyclosporine, tacrolimus, etc.), anti-proliferatives (e.g., azathioprine, mycophenolic acid, etc.), mTOR inhibitors (e.g., sirolimus, rapamycin, everolimus, etc.), and/or combinations thereof. In some embodiments, a short course of high-dose corticosteroids is applied and repeated. In some embodiments, corticosteroids are co-administered with one or more calcineurin inhibitors and one or more anti-proliferative agents. Alternatively or additionally, in some embodiments mTOR inhibitors are used where calcineurin inhibitors or steroids are contraindicated.

In some embodiments, antibody-based therapy includes administration of one or more antibodies or antibody-based drugs specific to select immune system components (e.g., IL-2Rα receptor, CD20, T-cells, etc.). Non-limiting examples of antibodies or antibody-based drugs include monoclonal anti-IL-2Rα receptor antibodies (e.g., Basiliximab, Daclizumab, etc.), polyclonal anti-T-cell antibodies (e.g., anti-thymocyte globulin [ATG], anti-lymphocyte globulin [ALG], etc.), monoclonal anti-T-cell antibodies (e.g., muromonab-CD3, Orthoclone OKT3, etc.), and monoclonal anti-CD20 antibodies (e.g., Rituximab).

Graft V Host Disease (GvHD)

Graft-versus-host disease (GvHD) is a common complication following an allogeneic or xenogenic tissue transplant (e.g. tissue implantation, graft, etc.). GvHD occurs when transplanted donor immune cells (e.g., white blood cells including T cells) present in the graft (i.e. donor) tissue recognize the host (i.e., recipient) as “non-self” (i.e., antigenically “foreign”) and attack the tissues of the recipient. Donor infection-fighting cells attack tissues in the host just as if they were attacking an infection. For example, in some embodiments, bone marrow transplant presents risk of GvHD, because mature donor lymphocytes present within transplanted donor marrow may recognize recipient tissues as foreign. The transplanted donor immune cells then attack the host's body cells and destroy them. GvHD is most commonly associated with stem cell or bone marrow transplant, but applies to other forms of tissue graft as well.

In some embodiments, three criteria must be met in order for GvHD to occur. One, an immune-competent graft (i.e., donor tissue) is administered (i.e., transplanted to host), with viable and functional donor immune cells. Two, the host is immunologically disparate (i.e., histo-incompatible). Three, the host is immune-compromised and cannot destroy or inactivate the transplanted donor immune cells.

After bone marrow transplantation, lymphocytes (i.e., T cells) present in the graft attack the tissues of the transplant host after perceiving host tissues as antigenically foreign. The donor lymphocytes produce an excess of cytokines, including TNF-alpha (TNF-α) and interferon-gamma (IFNγ).

A wide range of host antigens can initiate GvHD, among them human MHCs. However, GvHD can occur even when MHC-identical siblings are donors. MHC-identical siblings or MHC-identical unrelated donors often have genetically different proteins (i.e., minor histocompatibility antigens) that can be presented by MHC molecules to the donor's T-cells, which see these antigens as foreign and so mount an immune response.

While donor T-cells are undesirable as effector cells of GvHD, they are valuable for engraftment by preventing the host residual immune system from rejecting the bone marrow graft (i.e., host-versus-graft; discussed above).

In some embodiments, GvHD is either acute GvHD (aGVHD) or chronic GvHD (cGVHD). Acute GvHD (aGvHD) usually occurs within the first three months following a transplant, and can affect the skin, liver, stomach, and/or intestines.

In some embodiments, symptoms of aGvHD include rash, yellow skin and eyes due to elevated concentrations of bilirubin, and diarrhea. Acute GvHD is graded on a scale of 1 to 4; grade 4 is the most severe. In some embodiments, aGvHD can be fatal.

Chronic GvHD (cGvHD) is the late form of the disease, and usually develops three months or more after a transplant. The symptoms of cGvHD resemble spontaneously occurring autoimmune disorders such as lupus or scleroderma.

In some embodiments, GvHD is more easily prevented than treated. Preventive measures include the administration of cyclosporin with or without methotrexate or steroids after stem cell transplant. Alternatively or additionally, in some embodiments T lymphocytes are removed from the stem cell graft before it is transplanted.

In some embodiments, first-line treatment of GvHD includes steroid therapy. In some embodiments, chronic GvHD occurs approximately in 10-40 percent of patients after stem cell transplant. Symptoms vary more widely than those of acute GvHD and are similar to various autoimmune disorders. In some embodiments, symptoms include dry eyes, dry mouth, rash, ulcers of the skin and mouth, joint contractures (i.e., inability to move joints easily), abnormal blood test results of liver function, stiffening of the lungs (i.e., difficulty in breathing), inflammation in the eyes, difficulty in swallowing, muscle weakness, a white film in the mouth, and/or combinations thereof. In some embodiments, the incidence of GvHD increases with increasing degree of mismatch between donor and host HLA antigens, increasing donor age and increasing host age.

Post-Transplant Lymphoproliferative Disorder (PTLD)

Therapeutic immunosuppression after tissue and/or organ transplantation (discussed above) may result in uncontrolled B-cell proliferation and is referred to as post-transplant lymphoproliferative disorder (PTLD). In some embodiments, PTLD is an uncontrolled proliferation of B-cell lymphocytes in transplantation subjects following infection with Epstein-Barr virus. Patients may develop infectious mononucleosis-like lesions or polycolonal B-cell hyperplasia. In turn, some of these hyper-proliferative B-cells may undergo mutations which render them malignant, and give rise to a lymphoma.

### Graft V Tumor (GvT)

Bone marrow transplantation is frequently used to treat cancer (e.g., leukemias). In addition to the undesirable GvHD aspects of T-cell physiology discussed above, donor T-cells have proven to have a valuable “graft-versus-tumor” (GvT) effect wherein the donor's T-cells may recognize residual cancer cells (e.g., leukemia, lymphoma, etc.) as being different and destroy them. For example, in some embodiments patients who develop acute or chronic GvHD have lower disease (e.g., cancer) recurrence rates than patients who do not develop GvHD. In some embodiments, allogeneic transplantation of donor hematopoietic cells is an effective treatment of leukemia (graft-versus-leukemia or “GvL” effect), but the beneficial effect is limited by GvHD. In some embodiments, depletion of T-cells abrogates GvHD and GvL effects. In some embodiments, allogeneic GvT and GvL effects vary from one disease to another, the stage of the disease, donor histocompatibility, degree of chimerism, etc.

### Tissue Component Compositions

Among other things, the present invention provides tissue component compositions comprising or consisting of encapsulated tissue components. In some embodiments, provided tissue component compositions comprise or consist of nanoparticles encapsulating tissue components from an external source (e.g., donor tissue components) that is or will be transplanted into the host/recipient. In some embodiments, provided nanoparticle compositions comprise or consist of nanoparticles encapsulating host/recipient tissue components. In some embodiments, one or more microbial cells may be used with (or instead of) polymeric nanoparticles.

Nanoparticles

In some embodiments, the present invention provides compositions and methods for treating or preventing undesirable immune response to non-self tissue components in a subject by administering nanoparticles that encapsulate donor or host tissue components of interest. By using nanoparticles to deliver tissue components (e.g., MHC polypeptides), acute exposure of the unmatched MHC polypeptides to the subject's immune response is reduced or eliminated.

Nanoparticles useful in accordance with the present invention include those in which the nanoparticles are comprised of at least one polymer assembled into a micelle that bounds an interior lumen and has an external surface. In some embodiments, nanoparticles are comprised of at least one polymer that is a homopolymer, a diblock polymer, a triblock polymer, a multiblock copolymer, a linear polymer, a dendritic polymer, a branched polymer, a random block, etc., or combinations thereof. In some embodiments, nanoparticles are comprised of a blend and/or mixture of polymers.

In some embodiments, nanoparticles are comprised of one or more biocompatible polymers and/or one or more biodegradable polymers. In some embodiments, nanoparticles are comprised of one or more synthetic polymers, or derivatives thereof. In some embodiments, nanoparticles are comprised of one or more natural polymers, or derivatives thereof. In some embodiments, nanoparticles are comprised of combinations of synthetic and natural polymers, or derivatives thereof.

In some embodiments, nanoparticles are comprised of one or more polymers selected from the group consisting of poly(hydroxy acids) such as poly(lactic acid), poly(glycolic acid), poly(lactic acid-co-glycolic acid), poly(lactic-co-glycolic acid), and derivatives of poly(lactic-co-glycolic acid), PEGylated poly(lactic-co-glycolic acid), poly(lactide), poly(glycolide), poly(lactide-co-glycolide), poly(anhydrides), PEGylated poly(anhydrides), poly (ortho esters), derivatives of poly(ortho esters), PEGylated poly(ortho esters), poly(caprolactones), derivatives of poly(caprolactone), PEGylated poly(caprolactones), polyamines (e.g. spermine, spermidine, polylysine, and derivatives thereof), PEGylated polylysine, polyamides, polycarbonates, polypropylene fumarates), polyamides, polyphosphazenes, polyamino acids, polyethers, polyacetals, polylactides, polyhydroxyalkanoates, polyglycolides, polyketals, polyesteramides, poly(dioxanones), polyhydroxybutyrates, polyhydroxyvalyrates, polycarbonates, polyorthocarbonates, poly(vinyl pyrrolidone), polycyanoacrylates, polyalkylene oxalates, polyalkylene succinates, poly(malic acid), poly(methyl vinyl ether), poly(ethylene imine), poly(acrylic acid), poly(maleic anhydride), poly(ethylene imine), derivatives of poly(ethylene imine), PEGylated poly(ethylene imine), poly(acrylic acid), derivatives of poly(acrylic acid), PEGylated poly(acrylic acid), poly(urethane), PEGylated poly(urethane), derivatives of poly(urethane), poly(lactide), poly(glycolide), poly(hydroxy acids), polyesters, poly(arylates), polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone) and/or derivatives thereof.

In some embodiments, nanoparticles are comprised of one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and/or combinations thereof.

In some embodiments, nanoparticles are comprised of one or more natural polymers. Exemplary natural polymers include, but are not limited to, proteins (such as albumin, collagen, gelatin), prolamines (for example, zein), polysaccharides (such as alginate), cellulose derivatives (such as hydroxypropyl cellulose, sodium carboxymethyl cellulose, ethyl cellulose and cellulose acetate), polyhydroxyalkanoates (for example, polyhydroxybutyrate), and/or combinations thereof. In some embodiments, a natural polymer may comprise or consist of chitosan.

In some embodiments, nanoparticles are comprised of one or more polymers such as poly(lactide-co-glycolide) copolymerized with polyethylene glycol (PEG). Without wishing to be bound by any particular theory, it is proposed that arrangement of a nanoparticle so that PEG is exposed on the external surface, may increase stability of the nanoparticle in blood, perhaps at least in part due to the hydrophilicity of PEG.

In some embodiments, nanoparticles are comprised of PLGA.

In some embodiments, nanoparticles utilized in accordance with the present invention are as described in one or more of U.S. Pat. No. 7,534,448, U.S. Pat. No. 7,534,449, U.S. Pat. No. 7,550,154, US20090239789A1, US20090269397A1, US20100104503A1, US20100151436A1, US20100284965A1, WO2006080951, WO2008115641, WO2008109347, WO2009094273, WO2012167261 and WO2013003157.

In general, a nanoparticle is or comprises a particle having a diameter (e.g., average diameter) of less than 1000 nanometers (nm). In some embodiments, provided tissue component compositions comprise a population of nanoparticles. In some embodiments, a population of nanoparticles comprises nanoparticles of a uniform size. In some embodiments, a population of nanoparticles comprises nanoparticles of different sizes; in some embodiments showing a particular size distribution. In many embodiments, provided tissue component compositions comprise nanoparticles having sizes (e.g., average sizes) within a range defined by a lower limit and an upper limit. In some embodiments, the lower limit is 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, or more. In some embodiments, the upper limit is 1000 nm, 950 nm, 900 nm, 850 nm, 800 nm, 750 nm, 700 nm, 650 nm, 600 nm, 550 nm, 500 nm, 450 nm, 400 nm, 350 nm, 300 nm, 250 nm or less. In some embodiments, provided tissue component compositions comprise nanoparticles having sizes (e.g., average sizes) similar to the size of bacterial cells. For example, in some embodiments, provided tissue component compositions comprise nanoparticles having sizes (e.g., average sizes) ranging between 100 nm and 2000 nm, between 100 nm and 1000 nm, between 100 nm and about 500 nm, between 100 nm and about 300 nm, or between 100 nm and about 200 nm.

In some embodiments, provided tissue component compositions are substantially free of particles larger than about 2000 nm, about 1000 nm, about 900 nm, about 800 nm, about 700 nm, about 600 nm, about 500 nm, about 400 nm, or about 300 nm. In some embodiments, provided tissue component compositions comprise no more than about 50%, about 25%, about 10%, about 5%, or about 1% of particles larger than about 2000 nm, about 1000 nm, about 900 nm, about 800 nm, about 700 nm, about 600 nm, about 500 nm, about 400 nm, or about 300 nm.

Nanoparticles—Exemplary Methods of Making

In another aspect, the present invention provides methods of producing nanoparticles. In some embodiments, for example, embodiments wherein the nanoparticles include one or more of donor tissue component(s) and/or host tissue component(s), provided methods of making nanoparticles may include one or more of separating, associating, forming, emulsions, hot melt microencapsulation, solvent removal, spray-drying, and/or ionic gelation steps, and combinations thereof. Additionally, in some embodiments, provided nanoparticles comprise or are co-administered with one or more targeting components, for example, one or more adjuvants and/or components described in the section entitled “Other Components” below. For example, in some embodiments, provided nanoparticles comprise one or more of lipopolysaccharide and/or CpG motifs. Non-limiting exemplary methods of incorporating targeting components may be found in U.S. Pat. Nos. 7,534,448 and 7,534,449, the disclosures of which are hereby incorporated in their entirety.

Forming

In some embodiments, provided nanoparticles may be formed using any available method in the art. In some embodiments, provided nanoparticles and/or tissue component compositions may be prepared by nanoprecipitation, flow focusing using fluidic channels, spray drying, single and double emulsion solvent evaporation, solvent extraction, phase separation, hot melt microencapsulation, milling, microemulsion procedures, microfabrication, nanofabrication, sacrificial layers, simple and complex coacervation, and other methods well known to those of ordinary skill in the art. In some embodiments, provided tissue component compositions are prepared by aqueous and organic solvent syntheses (see for example, Pellegrino et al., 2005, Small, 1:48; Murray et al., 2000, Ann. Rev. Mat. Sci., 30:545; and Trindade et al., 2001, Chem. Mat., 13:3843). In some embodiments, provided tissue component compositions are prepared by nanoprecipitation or spray drying. Conditions used in preparing particles may be altered to yield particles of a desired size or property (e.g., hydrophobicity, hydrophilicity, external morphology, “stickiness,” shape, etc.). In general, methods of preparing nanoparticles and/or conditions used (e.g., solvent, temperature, concentration, air flow rate, etc.) may depend on identity of functional elements (e.g., tissue components) associated with the particles and/or the composition of the polymer matrix.

In some embodiments, additional methods for making nanoparticles for delivery of encapsulated agents are described in the literature (see for example, Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755).

Methods of Making—with Tissue Components

In some embodiments, provided methods further include a step of associating one or more tissue component(s) with a nanoparticle. Suitable tissue component(s) may include those described herein. In some embodiments, tissue components comprise intact tissue samples, e.g., obtained from an organism or from an organism's cells. In some embodiments, tissue components comprise cells of a relevant type as found in a tissue of interest. In some embodiments, tissue components comprise cells obtained from a tissue sample. In some embodiments, tissue components comprise some or all contents of one or more cells. In some embodiments, tissue components comprise living cells. In some embodiments, tissue components comprise dead or killed cells. In some embodiments, tissue components comprise disrupted cells. In some embodiments, tissue components comprise cell parts less than complete cells. In some embodiments, tissue components comprise a cellular extract, which may be or comprise a cellular lysate. In some embodiments, tissue components are provided in a crude sample, for example, that has been subject to little or no processing beyond separation from its source (e.g., as a primary tissue explant, cell sample, etc.). In some embodiments, tissue components are provided in a purified or processed sample, for example, that was subjected to one or more isolation, purification, and/or processing steps. In some embodiments, tissue components are or comprise substantially pure or purified entities. In some embodiments, tissue components are or comprise one or more entities that has/have been artificially produced in a system other than a natural organism. In some embodiments, tissue components comprise or consist of MHC molecules, which, in some embodiments, may be complexed with peptide(s) found in the tissue. In some such embodiments, such complexes are arranged, constructed, and/or assembled so that the peptide(s) is/are presented to relevant immune cells (e.g., T cells, B cells).

In some embodiments, provided methods further include a step wherein one or more tissue components are associated with either or both of the hydrophilic and/or hydrophobic cellular components so that some or all of the tissue component(s) is/are encapsulated within the internal lumen. In some embodiments, one or more tissue components are associated with the hydrophilic cellular components so that some or all of the tissue components(s) is/are encapsulated within the internal lumen. In some embodiments, one or more tissue components(s) are associated with the hydrophobic cellular components so that some or all of the tissue components(s) is/are encapsulated within the internal lumen.

As a more detailed example of some embodiments only, the use of certain methods, such as double emulsion, hot melt encapsulation, solvent removal, spray-drying, and ionic gelation methods for forming nanoparticles are provided. Exemplary methods for forming nanoparticles may be found in Demento et al., “TLR9-Targeted Biodegradable Nanoparticles as Immunization Vectors Protect Against West Nile Encephalitis”, 2010, J. Immunol. 185:2989-2997; see also Demento et al., “Inflammasome-activating nanoparticles as modular systems for optimizing vaccine efficacy”, 2009, Vaccine 27(23): 3013-3021.

Emulsions

In some embodiments, a polymer is dissolved in a volatile organic solvent, such as methylene chloride. The payload (for example, tissue components and/or MHC antigens) is added to the solution, and the mixture is suspended in an aqueous solution that contains a surface active agent such as poly(vinyl alcohol). The resulting emulsion is stirred until most of the organic solvent evaporates, leaving solid nanoparticles. The resulting nanoparticles are washed with water and dried overnight in a lyophilizer. Freeze dried nanoparticles may then be stored at −20° C. for later use.

In some embodiments, a water-in-oil-in-water (W/O/W) emulsion method may be used for preparation of the nanoparticles. In some embodiments, the nanoparticles include one or more hydrophilic cellular components. For example, in a first emulsion (W/O), aqueous cellular components in phosphate-buffered saline (PBS) are added to a vortexing PLGA solution dissolved in methylene chloride. The first emulsion of polymer and aqueous cellular lysate are then added drop-wise to PVA in a second emulsion (W/O/W). After each emulsion, samples are sonicated for 30 seconds on ice. The second emulsion is then rapidly added to 0.3% PVA. This external phase is then vigorously stirred for 3 hours at constant room temperature to evaporate the methylene chloride, leaving solid nanoparticles. Particles are collected by centrifugation. The resulting nanoparticles are washed with deionized water, flash-frozen, lyophilized, and stored at −20° C. for later use.

In some embodiments, the nanoparticles include one or more hydrophobic cellular components. The hydrophobic cellular component(s) are first combined with a second emulsion. The first emulsion of polymer (with or without aqueous cellular lysate and/or antigen) is then added drop-wise to the second emulsion (W/O/W).

In some embodiments, the nanoparticles further include one or more encapsulated host or donor antigens (for example, MHC antigens). In a first emulsion (W/O), concentrated antigen in phosphate-buffered saline (PBS) is added to a vortexing PLGA solution dissolved in methylene chloride. In some embodiments an aqueous cellular lysate is combined with the first emulsion. Polymer and encapsulant are then added drop-wise to a second emulsion (W/O/W). In some embodiments, the second emulsion has been combined with one or more hydrophobic cellular components. After each emulsion, samples are sonicated for 30 seconds on ice. The second emulsion is then rapidly added to 0.3% PVA. This external phase is then vigorously stirred for 3 hours at constant room temperature to evaporate the methylene chloride, leaving solid nanoparticles. Particles are collected by centrifugation. The resulting nanoparticles are washed with deionized water, flash-frozen, lyophilized, and stored at −20° C. for later use.

Hot Melt Microencapsulation

In this method, the polymer is first melted and then mixed with the solid particles. The mixture is suspended in a non-miscible solvent (like silicon oil), and, with continuous stirring, heated to a temperature, for example, 5° C., above the melting point of the polymer. Once the emulsion is stabilized, it is cooled until the polymer particles solidify. The resulting nanoparticles are washed by decantation with petroleum ether to give a free-flowing powder. Nanoparticles with sizes between 0.5 to 1000 microns may be obtained with this method. The external surfaces of nanoparticles prepared with this technique are usually smooth and dense. This procedure is used to prepare nanoparticles made of polyesters and polyanhydrides. In some embodiments, such a method may use polymers with molecular weights between 1,000-50,000.

Solvent Removal

This technique is primarily designed for polyanhydrides according to known methods. In some embodiments, a payload to be encapsulated (for example, tissue components and/or HLA antigens) is dispersed or dissolved in a solution of the selected polymer in a volatile organic solvent like methylene chloride. This mixture is suspended by stirring in an organic oil (such as silicon oil) to form an emulsion. Unlike solvent evaporation, this method may be used to make nanoparticles from polymers with high melting points and different molecular weights. The external morphology of nanoparticles produced with this technique is highly dependent on the type of polymer used.

Spray-Drying

In some embodiments using this method, the polymer is dissolved in organic solvent. A known amount of the payload (for example, tissue components and/or MHC polypeptides) is suspended (insoluble extract) or co-dissolved (soluble extract) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm.

Ionic Gelation

In some embodiments, such as those including nanoparticles made of gel-type polymers, such as alginate, traditional ionic gelation techniques may be used. Typically, the polymer(s) are first dissolved in an aqueous solution, mixed with barium sulfate or some bioactive agent, and then extruded through a nanodroplet forming device, which in some instances employs a flow of nitrogen gas to break off the droplet. A slowly stirred (approximately 100-170 RPM) ionic hardening bath is positioned below the extruding device to catch the forming nanodroplets. The nanoparticles are left to incubate in the bath for twenty to thirty minutes in order to allow sufficient time for gelation to occur. Nanoparticle size is controlled by using various size extruders or varying either the nitrogen gas or polymer solution flow rates. Chitosan nanoparticles can be prepared by dissolving the polymer in acidic solution and crosslinking it with tripolyphosphate. Carboxymethyl cellulose (CMC) nanoparticles can be prepared by dissolving the polymer in acid solution and precipitating the nanoparticle with lead ions. In the case of negatively charged polymers (e.g., alginate, CMC), positively charged ligands (e.g., polylysine, polyethyleneimine) of different molecular weights can be ionically attached.

Microbial Cells

In some embodiments, microbial cells may be used to encapsulate tissue components within provided tissue component compositions. In some embodiments, microbial cells are comprised of living cells; in some embodiments, microbial cells are comprised of dead (e.g., killed) cells.

In some embodiments, the present invention provides compositions and methods for treating or preventing undesirable immune response to non-self tissue components (e.g., MHC polypeptides) in a subject by administering modified bacterial, fungal, archaeal and/or protozoan cells (“microorganisms”) that express donor or host tissue components of interest. By using genetically modified microorganisms to express and deliver tissue components (e.g., MHC polypeptides), acute exposure of the unmatched MHC polypeptides to the subject's immune response is reduced or eliminated.

Without wishing to be bound by any particular theory, and without limitation to the mechanisms proposed, it is expected that the modified microorganisms and/or nanoparticles of the present invention are engulfed by antigen-presenting cells (APCs) such as macrophages and dendritic cells without exposing foreign tissue components (e.g., MHC polypeptides) to host antibodies. Once inside the APCs, the expressed tissue components are released by lysis of the microorganisms or secretion of the tissue components by the microorganisms. The tissue components are then processed, for example through partial digestion by the APCs, and displayed on the cell surface.

Once the processed tissue components are displayed on the cell surface, activation of the cytotoxic T cell response and helper T cell response promotes cellular immune response and Th1-mediated B cell response to microbial-expressed tissue components. Without wishing to be held to a particular theory, in some embodiments, this mechanism is expected to be sufficient to induce immune tolerance to one or more of the processed tissue components.

Any microorganism capable of expressing one or more tissue components may be used as delivery vehicles in accordance with the present invention. Such microorganisms include but are not limited to bacteria, viruses, fungi (including yeast), archaea (e.g., algae) and protozoa. Generally, microorganisms are single cell, single spore or single virion organisms. Additionally, included within the scope of the present invention are cells from multi-cellular organisms which have been modified to produce a polypeptide of interest. In some embodiments, microorganisms that can be genetically manipulated to produce a desired polypeptide are preferred (Ausubel et al. Current Protocols in Molecular Biology. Wiley and Sons, Inc. 1999, incorporated herein by reference). Genetic manipulation includes mutation of the host genome, insertion of genetic material into the host genome, deletion of genetic material of the host genome, transformation of the host with extrachromosomal genetic material, transformation with linear plasmids, transformation with circular plasmids, insertion of genetic material into the host (e.g., injection of mRNA), insertion of transposons, and chemical modification of genetic material. Methods for constructing nucleic acids (including an expressible gene), and introducing such nucleic acids into an expression system to express the encoded protein are well established in the art (see, for example, Sambrook et al., Molecular Cloning: A Laboratory Manual, 2nd Ed., Cold Spring Harbor Laboratory Press, Cold Spring Harbor, N. Y., 1989).

In general, any tissue component, such as MHC proteins or polypeptides may be produced by microorganisms in accordance with the present invention. Those skilled in the art are capable of identifying transfection and/or transduction techniques to introducing genetic material (e.g., plasmid DNA) into microorganisms to express proteins in accordance with the present invention. Non-limiting examples include transfection using calcium phosphate, cationic polymers (e.g., DEAE-dextran, polyethylenimine “PEI”), by electroporation, gene gun (where DNA is coupled to a nanoparticle of inert solid, e.g., gold, which is then “shot” directly into the target cell's nucleus), viral transduction (using virus as a carrier to introduce DNA into cell), heat shock, nucleofection, or by mixing a cationic lipid with genetic material to produce liposomes, which fuse with cell membranes an deposit cargo (i.e., genetic material) inside.

In some embodiments, bacteria are used as protein delivery microorganisms. Generally, bacteria are classified as gram-negative or gram-positive depending on the structure of the cell walls. Those skilled in the art are capable of identifying gram-negative and gram-positive bacteria which may be used to express proteins in accordance with the present invention. In some embodiments, bacteria for use in accordance with the present invention include, but are not limited to Actinomyces, Aeromonas, Anabaena, Arthrobacter, Bacillus, Bacteroides, Bdellovibrio, Bordetella, Borrelia, Campylobacter, Caulobacter, Chlamydia, Chlorobium, Chromatium, Citrobacter, Clostridium, Corynebacterium, Cytophaga, Deinococcus, Enterobacter, Escherichia, Francisella, Halobacterium, Heliobacter, Haemophilus, Hemophilus influenza type B (HIB), Hyphomicrobium, Klebsiella, Lactococcus, Legionella, Leptspirosis, Listeria, Meningococcus A, B and C Methanobacterium, Micrococcus, Morganella, Myobacterium, Mycoplasma, Myxococcus, Neisseria, Nitrobacter, Oscillatoria, Peptococcus, Phodospirillum, Plesiomonas, Prochloron, Proteus, Providencia, Pseudomonas, Rickettsia, Salmonella, Serratia, Shigella, Spirillum, Spirochaeta, Sporolactobacillu, Staphylococcus, Streptococcus, Streptomyces, Sulfolobus, Thermoplasma, Thiobacillus, and Treponema, Vibrio, Yersinia, and combinations thereof. In some embodiments, E. coli cells are a preferred delivery microorganism.

In some embodiments, fungi (including yeast) are used as protein delivery microorganisms. Those skilled in the art are capable of identifying fungi which may be used to express proteins in accordance with the present invention. In some embodiments, fungi for use in accordance with the present invention include, but are not limited to Brettanomyces anomalus, Brettanomyces bruxellensis, Brettanomyces claussenii, Brettanomyces custersianus, Brettanomyces lambicus, Brettanomyces naardenensis, Brettanomyces nanus, Canida albicans, Candida blankii, Candida slooffi, Dekkera intermedia, Leucosporidium frigidum, Rhodotorula rubra, Saccharomyces cerevisiae, Saccharomyces pastorianus, Saccharomyces telluris, Schizosaccharomyces pombe, Sporidiobolus johnsonii, Sporidiobolus longiusculus, Sporidiobolus metaroseus, Sporidiobolus pararoseus, Sporidiobolus ruineniae, Sporidiobolus salmonicolor, Sporidiobolus veronae, Trichosporon beigelii, Trichosporon cutaneum, and combinations thereof. In some embodiments, S. cerevisiae cells are a preferred delivery microorganism.

In some embodiments, archaea are used as protein delivery microorganisms. Those skilled in the art are capable of identifying archaea which may be used to express proteins in accordance with the present invention. In some embodiments, archaea for use in accordance with the present invention include, but are not limited to Aeropyrum pernix, Cenarchaeum symbiosum, Halobacterium salinarum, Halorubrum salsolis, Methanobrevibacter smithii, Methanogenium boonei, Methanosarcina acetivorans, Methanothrix, Nanoarchaeum equitans, Nitrosopumilus maritimus, Nitrososphaera gargensis, Nitrososphaera viennensis, Pyrococcus furiosus, Pyrolobus fumarii, Thermococcus celer, Thermococcus gammatolerans, Thermococcus kodakarensis, Thermococcus litoralis, and combinations thereof.

In some embodiments, protozoa are used as protein delivery microorganisms. Those skilled in the art are capable of identifying protozoa which may be used to express proteins in accordance with the present invention. In some embodiments, protozoa for use in accordance with the present invention include, but are not limited to Amoeboids (e.g., Entamoeba histolytica), Ciliates (e.g., Balantidium coli), Flagellates (e.g., Giardia lamblia), Sporozoans (e.g., Plasmodium knowlesi), and combinations thereof.

Microorganisms of the present invention may be administered to a subject as live or dead microorganisms. Preferably if the microorganisms are administered as live microorganisms, they are non-pathogenic or attenuated pathogenic microorganisms. For applications of the invention where live microorganisms are administered to individuals, preferably the microorganisms are attenuated and/or are administered in suitable encapsulation materials and/or as pharmaceutical compositions as vaccines to decrease an individual's immune response to the microorganism and/or allergenic compounds. Generally, attenuation involves genetically modifying the infectious pathogenic microorganism to reduce or eliminate the infectious ability of the microorganism. Preferably, the microorganism is attenuated such that an individual inoculated with the microorganism does not suffer any cytotoxic effects from the presence of the microorganism. Particularly preferred attenuated microorganisms are infectious intracellular pathogens which are phagocytosed by antigen-presenting cells in individuals who are exposed to the microorganism. Examples of microorganisms which are intracellular pathogens include Salmonella, Mycobacterium, Leishmania, Legionella, Listeria, and Shigella.

Microorganisms of the present invention may be administered to subjects after killing the microorganisms. Any method of killing the microorganisms may be utilized that does not greatly alter the antigenicity of the expressed polypeptides. Methods of killing microorganism include but are not limited to using heat, antibiotics, chemicals such as iodine, bleach, ozone, and alcohols, radioactivity (i.e. irradiation), UV light, electricity, and pressure. Preferred methods of killing microorganisms are reproducible and kill at least 99% of the microorganisms. Particularly preferred is the use of heat above 50 degrees Celsius for a period of time that kills greater than 99% of the cells and preferably 100% of the cells.

In some embodiments, expression of target tissue components (e.g., MHC proteins and/or polypeptides) by microorganisms is regulated so that synthesis occurs at a controlled time after the live microorganism is administered to an individual. Preferably the induction of protein synthesis is regulated so that activation occurs after the microorganism(s) is taken up by antigen-presenting cells (APCs) and phagocytosed into the endosome. A desirable result of this regulation is that production of the tissue components of interest occurs inside the APCs and therefore reduces or eliminates the (premature) exposure of the foreign tissue components to an individual's immune system. This reduces or eliminates the risk of deleterious host immune response during administration of microorganisms that produce foreign tissue components.

Any method of controlling protein synthesis in the microorganism may be used in accordance with the present invention. In some embodiments, the method of controlling protein synthesis utilizes an inducible promoter operatively-linked to the gene of interest (e.g., a gene which encodes a signal peptide and protein antigen). Many systems for controlling transcription of a gene using an inducible promoter are known (Ausubel et al. Current Protocols in Molecular Biology. Wiley and Sons. New York. 1999). Generally, inducible systems either utilize activation of the gene or derepression of the gene. In some embodiments, it is preferred that the present invention utilizes activation of a gene to induces transcription. However, inducible systems using derepression of a gene may also be used. Systems using activation are preferred in some embodiments because these systems are able to tightly control inactivation (and hence basal level synthesis) since derepression may result in low levels of transcription if the derepression is not tight.

Methods of inducing transcription include, but are not limited to, induction by the presence or absence of a chemical agent, induction using a nutrient starvation inducible promoter, induction using a phosphate starvation inducible promoter and induction using a temperature sensitive inducible promoter. A particularly preferred system for regulating gene expression utilizes tetracycline controllable expression system. Systems which utilize the tetracycline controllable expression system are commercially available (see for example, Clontech, Palo Alto, Calif.).

It is preferred that inducible systems for use in the present invention utilize inducing agents that are non-toxic to mammalians cells including humans. Furthermore, it is preferred that transcriptional inducing agents permeate cells membranes. More specifically for activation of protein synthesis in microorganisms after phagocytosis by APCs, transcriptional inducing agents must be able to pass through cells membranes of the APC and cell membranes of the microorganism to activate the expression of genes encoding protein allergens in accordance with the present invention. Since both tetracycline and ecdysone are able to pass through cell membranes and are non-toxic, tetracycline-inducible systems and ecdysone-inducible systems are ideally suited for use in some embodiments of the present invention. However, the use of inducible systems is not limited to those systems.

It is also preferred that bacteria that have not been phagocytosed are killed before induction of genes expressing polypeptide allergens of interest. A preferred method of killing bacteria is to use antibiotics which are not permeable to mammalian cell membranes such that only bacteria that are not phagocytosed are killed. The use of antibiotics in accordance with the present embodiment reduces or eliminates the production of polypeptides by bacteria outside antigen presenting cells. According to various embodiments, it is important to reduce or eliminate exposure of allergen-producing bacteria to the immune system, especially bacteria that secrete polypeptides, which could elicit a potentially lethal anaphylactic reaction in an individual. Those having ordinary skill in the art are readily aware of antibiotics which may be used. Such antibiotics include but are not limited to penicillin, ampicillin, cephalosporin, griseofulvin, bacitracin, polymyxin b, amphotericin b, erythromycin, neomycin, streptomycin, tetracycline, vancomycin, gentamicin, rifamycin and combinations thereof.

Tissue Components

As described herein, the present invention encompasses the recognition that certain advantages are achieved when host and/or donor tissue components are encapsulated to create tissue component formulations of the present invention. In some embodiments, the present invention provides tissue components—e.g., intact or disrupted tissue materials for use in or with tissue component formulations. In some embodiments, tissue components comprise or consist of MHC molecules, which in some embodiments may be complexed with peptide(s) found in the tissue. In some such embodiments, such complexes are arranged, constructed, and/or assembled so that the peptide(s) is/are presented to relevant immune cells (e.g., T cells, B cells).

As will be appreciated by those skilled in the art, reading the present disclosure, in some embodiments the present invention provides transfer of one or more tissue components from a donor source into a recipient (host) individual. Non-limiting examples of tissues include stem cells, bone and bone marrow, heart and heart valves, liver, kidney, lung, pancreas, small intestine, cornea, tendons, cartilage, connective tissue, skin, blood and vessels. For example, in some embodiments, arteries, veins and femoral vessels may be transplanted to patients with compromised blood circulation. In some embodiments, bone and ligament tissue may be transplanted to patients with bone or spinal injuries, partially or fully restoring function and preventing amputation. In some embodiments, corneas may be transplanted to patients to prevent blindness or restore sight. In some embodiments, heart valves may be transplanted to people with heart defects or disease. In some embodiments, skin transplants may be given to patients who have been severely burned or who have major skin loss from injury or disease.

As will be appreciated by those skilled in the art, reading the present disclosure, in some embodiments the present invention provides transplantation of one or more organs from a donor source into a recipient (host) individual. Non-limiting examples of organs include lung, heart, liver, pancreas, kidney and liver. For example, in some embodiments lung transplants may be performed in patients with damaged lung tissue (e.g., suffering from non-malignant pulmonary disease, emphysema, cystic fibrosis, pulmonary fibrosis or pulmonary hypertension, etc.). In some embodiments, two or more organs are transplanted from an external source into a recipient (host) individual (e.g., lung and heart transplant). Alternatively or additionally, in some embodiments the present invention provides transplantation of one or more body extremities (e.g., leg, foot, hand, arm) or structures (e.g., ear, nose, face). For example, in some embodiments hand transplants may be performed in patients who have suffered a below-elbow amputation. In another example, in some embodiments face transplants (i.e. replace all or part of a patients face) may be performed in patients who have suffered disfigurement by trauma, burns, disease, or birth defects.

As will be appreciated by those skilled in the art, reading the present disclosure, tissue components may be harvested from any donor source. In some embodiments, tissue components are harvested from a donor organism. In some embodiments, a donor organism survives after tissue components are removed (e.g., single kidney harvested; partial lung harvest, etc.). In some embodiments, tissue components are harvested from a donor who has been declared brain dead. In some embodiments, a dead donor is or has received mechanical ventilation to maintain (oxygenated) blood flow and tissue and/or organ viability post-death.

In some embodiments, a donor source for tissue components is tissue culture. In some embodiments, tissue culture is in vitro. In some embodiments, tissue culture is ex vivo. Alternatively or additionally, in some embodiments tissue components are grown and/or produced in tissue culture. For example, osteoblasts and osteoclasts may be grown in culture to produce bone for transplantation. In another example, cartilage cells may be cultured in tissue culture to produce cartilage for transplantation (e.g., an ear-shaped cartilage structure). In yet another example, embryonic and/or adult stem cells may be grown in tissue culture to various tissue components for transplantation. In some embodiments, embryonic and/or adult stem cells remain pluripotent (i.e., undifferentiated). In some embodiments, embryonic and/or adult stem cells are differentiated into specific tissue components including, but not limited to bone and bone marrow, heart and heart valves, liver, kidney, lung, pancreas, small intestine, cornea, tendons, cartilage, connective tissue, skin, blood, vessels and combinations thereof.

In some embodiments, tissue components comprise one or more MHC proteins. In some embodiments, tissue components comprise one or more MHC polypeptides.

In some embodiments, a host (i.e., recipient) may also serve as a donor source, i.e., host tissue components are combined with nanoparticles and/or microbial cells and transplanted back into the host.

In some embodiments, tissue components are encapsulated within a nanoparticle. In some embodiments, one or more MHC proteins are encapsulated within a nanoparticle and/or microbial cell. In some embodiments, one or more MHC polypeptides are encapsulated within a nanoparticle and/or microbial cell.

In some embodiments, encapsulated tissue components are from one or more donor organisms. In some embodiments, encapsulated tissue components are from the host organism.

Other Components

In some embodiments, the provided tissue component compositions and/or formulations may include one or more other agents (e.g. adjuvants). Without wishing to be held to a particular theory, it is possible that some embodiments may mimic one or more characteristics or features of microbial (e.g., bacterial) cells. In some embodiments, adjuvants may be provided from one or more bacterial sources, including bacterial cellular lysates and/or cellular lysate fractions. In some embodiments, bacterial cellular lysate fractions are or comprise entities known as pathogen-associated molecular patterns (“PAMPs”). In some embodiments, one or more of a hydrophobic bacterial cellular lysate fraction and/or hydrophilic bacterial cellular lysate fraction include one or more PAMPs as a hydrophilic cellular component and/or hydrophobic cellular component.

In some embodiments, PAMPs are entities associated with bacterial cells that are recognized by cells of the innate immune system. In some embodiments, PAMPs are recognized by Toll-like receptors (TLRs) and other pattern recognition receptors (PRRs) in both plants and animals. In some embodiments, PAMPs are recognized by C-type lectin receptors (CLRs). In some embodiments, a CLR is a type I or type II CLR. In some embodiments, PAMPs are or comprise entities associated with the outer surface of a bacterial cell, including, but not limited to, membrane-associated proteins and/or peptides, receptors embedded in bacterial membranes, etc. Exemplary PAMPs include, but are not limited to, bacterial lipopolysaccharide (LPS), bacterial flagellin, lipoteichoic acid from gram positive bacteria, peptidoglycan, double-stranded RNAs (dsRNAs), unmethylated CpG motifs, any of the TLR ligands presented in Table 1, characteristic portions thereof, and/or combinations thereof

TABLE 1
Exemplary TLRs and TLR Ligands

| TLR | TLR Ligand(s) |
|---|---|
| TLR1 | Multiple triacyl lipopeptides (e.g., from bacteria and |
| | mycobacteria), such as lipopeptide Pam3Cys-SK4 (“Pam”) |
| TLR2 | Multiple glycolipids, lipopeptides and lipoproteins, such as |
| | lipopeptide Pam3Cys-SK4 (“Pam”) |
| | Lipoteichoic acid |
| | Peptidoglycan |
| | HSP70 |
| | Zymosan |
| | Heat shock proteins, such as Hsp60 |
| TLR3 | Double-stranded RNA |
| | Single-stranded RNA |
| | Poly(I:C) |
| TLR4 | lipopolysaccharide (LPS) |
| | Monophosphoryl lipid A (MPL) |
| | Several heat shock proteins |
| | Fibrinogen |
| | Heparin sulfate fragments |
| | Hyaluronic acid fragments |
| TLR5 | Flagellin |
| TLR6 | Multiple diacyl lipopeptides |
| | Lipoteichoic acid (LTA) |
| | Zymosan |
| TLR7 | Imidazoquinolines (e.g., imiquimod and resiquimod) |
| | Single-stranded RNA, such as GU-rich single-stranded RNA |
| | Loxoribine (a guanosine analog) |
| | Bropirime |
| TLR8 | Imidazoquinolines (e.g., imiquimod and resiquimod) |
| | GU-rich single-stranded RNA |
| | Small synthetic compounds |
| | Single-stranded RNA |
| TLR9 | Unmethylated CpG DNA |
| | Hemazoin crystals |
| | Double-stranded DNA |
| TLR10 | |
| TRL11 | Toxoplasma gondii profilin |
| | Uropathogenic-bacteria-derived protein |

In some embodiments, the one or more other agents is or comprises one or more adjuvants. In some embodiments, an adjuvant is a mucosal adjuvant (i.e., an adjuvant capable of eliciting or enhancing an immune response to a mucosally administered tissue component). Exemplary mucosal antigens include, but are not limited to, TLR4 ligands (e.g. LPS, MPL), cytokines (e.g. IL-1α), c48/80, R848, Pam3CSK4, CpG(ODN1826), lethal factor (LF), and cholera toxin. It will be recognized by those of skill in the art that particular mucosal adjuvants may induce different immune responses. The skilled artisan will understand and be aware of technologies that may be used to select particular adjuvant(s) for use in a particular product or products and such variation is specifically contemplated as within the scope of the present invention.

One of skill in the art will recognize that multiple MHC proteins may be delivered by nanoparticles and/or microbial cells simultaneously and/or sequentially in accordance with methods of the present invention. Without limitation, different MHC molecules for one tissue component may be delivered. Different MHC molecules from different tissue components may also be delivered. Further, multiple MHC polypeptides and proteins may be delivered in accordance with the present invention. It is also recognized that single or multiple MHC polypeptides and single or multiple cytokines may be delivered to individuals by nanoparticles in accordance with the present invention. For example, but without limitation, MHC antigens of the present invention and immunomodulatory molecules such as interleukins may be delivered by nanoparticles using methods in accordance with the present invention.

In some embodiments, a particular provided composition may contain a combination of antigens other than MHC proteins. For example, in some embodiments, a particular provided composition may contain a combination of antigens associated with a particular disease, disorder or condition (e.g., with a particular cancer, a particular infectious disease, a particular graft v host or host v graft disease, etc).

Those of skill in the art will recognize a wide variety of potential applications utilizing combinations of tissue components and/or antigens; each of these is contemplated as within the scope of the present invention.

According to various embodiments, provided compositions comprising an antigen or other protein agent may comprise the antigen or other protein agent in any of a variety of forms. Exemplary forms include, without limitation, RNA, DNA, protein, and combinations thereof. In some embodiments, the antigen or protein agent may be provided as a portion of a cell, tissue or extract thereof.

In some embodiments, other agents include, but are not limited to, immunosuppressive agents such as steroids (e.g., prednisone and methylprednisolone), cyclophosphamide, cyclosporin A, FK506, thalidomide, azathioprine, monoclonal antibodies (e.g., Daclizumab (anti-interleukin (IL)-2), Infliximab (anti-tumor necrosis factor), MEDI-205 (anti-CD2), abx-cbl (anti-CD147)), and polyclonal antibodies (e.g., ATG (anti-thymocyte globulin)).

### Pharmaceutical Formulations

In some embodiments, the present invention provides pharmaceutical formulations comprising a provided tissue component composition together with one or more pharmaceutically acceptable excipients.

In some embodiments, provided pharmaceutical formulations may be prepared by any appropriate method, for example as known or hereafter developed in the art of pharmacology. In general, such preparatory methods include the step of bringing a provided tissue component composition into association with one or more pharmaceutically acceptable excipients, and then, if necessary and/or desirable, shaping and/or packaging the product into an appropriate form for administration, for example as or in a single- or multi-dose unit.

In some embodiments, formulations may be prepared, packaged, and/or sold in bulk, as a single unit dose, and/or as a plurality of single unit doses. As used herein, a “unit dose” is a discrete amount of the pharmaceutical formulation comprising a predetermined amount of the provided tissue component composition. The amount of the provided nanoparticle composition is generally equal to the dosage of the provided nanoparticle which would be administered to a subject and/or a convenient fraction of such a dosage such as, for example, one-half or one-third of such a dosage.

In many embodiments, provided pharmaceutical formulations are specifically formulated for mucosal delivery (e.g., oral, nasal, rectal or sublingual delivery).

In some embodiments, appropriate excipients for use in provided pharmaceutical formulations may, for example, include one or more pharmaceutically acceptable solvents, dispersion media, granulating media, diluents, or other liquid vehicles, dispersion or suspension aids, surface active agents and/or emulsifiers, isotonic agents, thickening or emulsifying agents, preservatives, solid binders, lubricants, disintegrating agents, binding agents, preservatives, buffering agents and the like, as suited to the particular dosage form desired. Alternatively or additionally, pharmaceutically acceptable excipients such as cocoa butter and/or suppository waxes, coloring agents, coating agents, sweetening, flavoring, and/or perfuming agents can be utilized. Remington's The Science and Practice of Pharmacy, 21^{st} Edition, A. R. Gennaro (Lippincott, Williams & Wilkins, Baltimore, Md., 2005; incorporated herein by reference) discloses various excipients used in formulating pharmaceutical formulations and known techniques for the preparation thereof.

In some embodiments, an appropriate excipient is at least 95%, at least 96%, at least 97%, at least 98%, at least 99%, or 100% pure. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or other International Pharmacopoeia.

In some embodiments, liquid dosage forms (e.g., for oral and/or parenteral administration) include, but are not limited to, emulsions, microemulsions, solutions, suspensions, syrups, and/or elixirs. In addition to provided tissue component compositions, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such a CREMOPHOR®, alcohols, oils, modified oils, glycols, polysorbates, cyclodextrins, polymers, and/or combinations thereof.

In some embodiments, injectable preparations, for example, sterile aqueous or oleaginous suspensions, may be formulated according to known methods using suitable dispersing agents, wetting agents, and/or suspending agents. Sterile liquid preparations may be, for example, solutions, suspensions, and/or emulsions in nontoxic parenterally acceptable diluents and/or solvents, for example, as a solution in 1,3-butanediol. Among the acceptable vehicles and solvents that may be employed, for example, are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of liquid formulations.

Liquid formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, one or more strategies may be utilized prolong and/or delay the effect of a provided pharmaceutical formulation after delivery.

In some embodiments, provided pharmaceutical formulations may be formulated as suppositories, for example for rectal or vaginal delivery. In some embodiments, suppository formulations can be prepared by mixing utilizing suitable non-irritating excipients such as cocoa butter, polyethylene glycol or a suppository wax which are solid at ambient temperature but liquid at body temperature and therefore melt in the body (e.g., in the rectum or vaginal cavity) and release the provided tissue component composition.

In some embodiments, solid dosage forms (e.g., for oral administration) include capsules, tablets, pills, powders, and/or granules. In such solid dosage forms, the provided tissue component composition may be mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

In some embodiments, solid formulations of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

In some embodiments, solid dosage forms may optionally comprise opacifying agents and can be of a composition that they release the provided tissue component composition(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In some embodiments, the present invention provides formulations for topical and/or transdermal delivery, e.g., as a cream, liniment, ointment, oil, foam, spray, lotion, liquid, powder, thickening lotion, or gel. Particular exemplary such formulations may be prepared, for example, as products such as skin softeners, nutritional lotion type emulsions, cleansing lotions, cleansing creams, skin milks, emollient lotions, massage creams, emollient creams, make-up bases, lipsticks, facial packs or facial gels, cleaner formulations such as shampoos, rinses, body cleansers, hair-tonics, or soaps, or dermatological compositions such as lotions, ointments, gels, creams, liniments, patches, deodorants, or sprays.

In some embodiments, an adjuvant is provided in the same formulation with provided tissue component composition(s) so that adjuvant and provided tissue component composition are delivered substantially simultaneously to the individual. In some embodiments, an adjuvant is provided in a separate formulation. Separate adjuvant may be administered prior to, simultaneously with, or subsequent to provided tissue component composition administration.

In some embodiments, provided formulations are stable for extended periods of time, such as 1 week, 2 weeks, 1 month, 2 months, 6 months, 1 year, 2 years, 3 years, or more. In some embodiments, provided formulations are easily transportable and may even be sent via traditional courier or other package delivery service. Such attributes may allow for rapid distribution of provided compositions to those in need.

In some embodiments, it may be advantageous to release encapsulated agent, for example, a tissue or tissue component(s), at various locations along a subject's gastrointestinal (GI) tract. In some embodiments, it may be advantageous to release encapsulated agent, for example, a donor tissue or tissue component(s), in a subject's mouth as well as one or more locations along the subject's GI tract. Accordingly, in some embodiments, a plurality of provided formulations (e.g. two or more) may be administered to a single subject to facilitate release of encapsulated agent at multiple locations. In some embodiments, each of the plurality of formulations has a different release profile, such as provided by various enteric coatings, for example. In some embodiments, each of the plurality of formulations has a similar release profile. In some embodiments, the plurality of formulations comprises one or more tissue or tissue components. In some embodiments, each of the plurality of administered formulations comprises a different tissue or tissue component(s). In some embodiments, each of the plurality of formulations comprises the same tissue or tissue component(s).

In some embodiments, one or more agents may be included that can affect rate and/or extent of release of agent (e.g., tissue or tissue component(s)) from nanoparticles and/or microbial cells. In some embodiments, such an agent would affect rate and/or extent of release by leakage or otherwise undesired release (e.g., at a site other than a target site and/or at a time other than a desired time). Without wishing to be bound by any particular theory, in some embodiments, such agents may coat or block release sites on nanoparticle and/or microbial surfaces. In some embodiments, such agents may be or comprise tannic acid.

### Routes of Administration

In some embodiments, provided tissue component compositions may be formulated for any appropriate route of delivery. In some embodiments, provided tissue component compositions may be formulated for any route of delivery, including, but not limited to, bronchial instillation, and/or inhalation; buccal, enteral, interdermal, intra-arterial (IA), intradermal, intragastric (IG), intramedullary, intramuscular (IM), intranasal, intraperitoneal (IP), intrathecal, intratracheal instillation (by), intravenous (IV), intraventricular, mucosal, nasal spray, and/or aerosol, oral (PO), as an oral spray, rectal (PR), subcutaneous (SQ), sublingual; topical and/or transdermal (e.g., by lotions, creams, liniments, ointments, powders, gels, drops, etc.), transdermal, vaginal, vitreal, and/or through a portal vein catheter; and/or combinations thereof. In some embodiments, the present invention provides methods of administration of provided tissue component compositions via mucosal administration. In some embodiments, the present invention provides methods of administration of provided tissue component compositions via oral administration. In some embodiments, the present invention provides methods of administration of provided tissue component compositions via sublingual administration.

In some embodiments, one or more provided compositions are administered intravenously, intradermally, transdermally, orally, subcutaneously, and/or transmucosally.

In some embodiments, mucosal (e.g., transmucosal) administration includes, but is not limited to, buccal, nasal, bronchial, vaginal, rectal and/or sublingual administration. In some embodiments, transmucosal administration is buccal, nasal, bronchial, vaginal, rectal, and/or sublingual administration.

### Uses/Methods of Treatment

The present invention provides, among other things, methods of treating and/or preventing graft rejection with provided compositions. In some embodiments, graft rejection may be autograft rejection, xenograft rejection, and/or allograft rejection.

Autograft Rejection

In some embodiments, graft rejection refers to an autograft rejection, wherein the donor individual and recipient individual are the same (i.e., a patient's own tissue).

The present invention provides, among other things, methods of administering to a recipient organism who has received or will receive a transplant of one or more heterologous tissue components from a donor organism, a composition comprising encapsulated recipient organism tissue components.

In some embodiments, one or more recipient tissue components are encapsulated within a nanoparticle. In some embodiments, one or more recipient tissue components are encapsulated within a microbial cell. In some embodiments, one or more provided compositions are administered prior to the transplant. Alternatively or additionally, in some embodiments, one or more provided compositions are administered subsequent to the transplant.

Xenograft Rejection

In some embodiments, graft rejection refers to a xenograft rejection, wherein the donor and recipient are of different species. Typically, xenograft rejection occurs when the donor species tissue carries a xenoantigen against which the recipient species immune system mounts a rejection response.

The present invention provides, among other things, methods of administering to a recipient organism who has received or will receive a transplant of one or more heterologous tissue components from a donor organism of different species a composition comprising encapsulated donor organism tissue components.

For example, in preparation for a heart valve transplant (e.g., from pig to human), donor porcine tissue components are isolated, encapsulated within nanoparticles, and administered to a recipient organism.

Allograft Rejection

In some embodiments, graft rejection refers to an allograft rejection, wherein the donor individual and recipient individual are of the same species. Typically, allograft rejection occurs when the donor tissue carries an alloantigen against which the recipient immune system mounts a rejection response.

The present invention provides, among other things, methods of administering to a recipient organism who has received or will receive a transplant of one or more heterologous tissue components from a donor organism a composition comprising encapsulated donor organism tissue components.

In some embodiments, one or more heterologous tissue components are encapsulated within a nanoparticle. In some embodiments, one or more heterologous tissue components are encapsulated within a microbial cell. In some embodiments, one or more heterologous tissue components are from a different species. In some embodiments, one or more provided compositions are administered prior to the transplant. Alternatively or additionally, in some embodiments, one or more provided compositions are administered subsequent to the transplant.

The present invention also provides, among other things, methods of administering to a donor organism from which one or more donor tissue components are to be transplanted into a recipient organism a composition comprising one or more encapsulated recipient tissue components.

In some embodiments, a donor organism is a different species that a recipient organism. In some embodiments, one or more encapsulated recipient tissue components are encapsulated within a nanoparticle. In some embodiments, one or more one or more encapsulated recipient tissue components are encapsulated within a microbial cell. In some embodiments, one or more provided compositions are administered prior to the transplant. Alternatively or additionally, in some embodiments, one or more provided compositions are administered subsequent to the transplant.

In another aspect, the present invention provides, among other things, a method comprising determining which MHC proteins are expressed by a donor organism; determining which MHC proteins are expressed by a recipient organism; selecting one or more encapsulated MHC proteins that matches one or more MHC proteins of the donor organism; and administering to the recipient organism into which one or more donor tissue components are to be transplanted from the donor organism the one or more encapsulated MHC proteins.

In some embodiments, the present invention provides methods of treating various immune-related diseases, disorders and/or conditions.

In some embodiments, provided formulations may be used to treat patients with various forms of GvHD including acute and chronic GvHD that is either naïve or refractory to conventional immunosuppressive agents such as steroids and cyclosporine A. In some embodiments, provided formulations may be used as prophylaxis to prevent onset of GvHD by pretreating (i.e., tolerizing) the transplant recipient (i.e. host) prior to the transplantation and/or treating the recipient (i.e., host) within a certain time window post transplantation. In some embodiments, provided formulations may be used to treat patients with various forms of HvGD.

Treatment of GvHD

In some embodiments, a method is provided for treating a patient suffering from GvHD, said method comprises administering to the GvHD patient one or more heterologous tissue components from a donor organism a composition comprising encapsulated donor organism tissue components.

In some embodiments, the host (e.g., transplant recipient) is pretreated with one or more provided formulations comprising donor tissue components. Alternatively or additionally, in some embodiments the host is pretreated with one or more provided formulations comprising host (i.e., self) tissue components.

In some embodiments, the host is treated with one or more provided formulations comprising host (i.e., self) tissue components after receiving one or more tissue transplantation (i.e., tissue graft, organ transplant, etc.). Without wishing to be held to a particular theory, it is expected that administration of host tissue to the host pre- and/or post-transplantation will decrease the host immune response to the transplanted tissue.

In some embodiments, the donor is pretreated with one or more provided formulations comprising host (i.e., transplant recipient) tissue components. Without wishing to be bound to any particular theory, the present invention proposes that the host could be tolerized to the donor and the donor could be tolerized to the host to provide effective “cross tolerization” to provide a better clinical outcome after the transplant. As a non-limiting example, in some embodiments, donor bone marrow is exposed to recipient antigens ex vivo prior to transplant.

Dosage amounts and frequency will vary according the particular formulation, the dosage form, and individual patient characteristics. Generally speaking, determining the dosage amount and frequency for a particular formulation, dosage form, and individual patient characteristic can be accomplished using conventional dosing studies, coupled with appropriate diagnostics.

GvHD Prophylaxis

Provided nanoparticle formulations can also be used as a prophylaxis to prevent onset of GvHD or to reduce the effects of GvHD. In some embodiments, provided formulations may be administered as a GvHD prophylaxis to a transplant recipient (i.e., host) within a predetermined time window before or after the transplantation.

In some embodiments, provided formulations may be administered to the host on days −3 or −2 (i.e., 3 or 2 days before the transplantation) as part of a tolerizing regiment, then followed by transplantation such as hematopoietic stem cell infusion.

In another embodiment, provided formulations may be administered as a GvHD prophylaxis to a transplant recipient after the transplantation.

Combination Therapy

In some embodiments, provided pharmaceutical formulations are administered to a subject in combination with one or more other therapeutic agents or modalities, for example, useful in the treatment of one or more diseases, disorders, or conditions treated by the relevant provided pharmaceutical formulation, so the subject is simultaneously exposed to both. In some embodiments, a provided tissue component composition is utilized in a pharmaceutical formulation that is separate from and distinct from the pharmaceutical formulation containing the other therapeutic agent. In some embodiments, a provided tissue component composition is admixed with the composition comprising the other therapeutic agent. In other words, in some embodiments, a provided tissue component composition is produced individually, and the provided tissue component composition is simply mixed with another composition comprising another therapeutic agent.

The particular combination of therapies (substances and/or procedures) to employ in a combination regimen will take into account compatibility of the desired substances and/or procedures and the desired therapeutic effect to be achieved. In some embodiments, provided formulations can be administered concurrently with, prior to, or subsequent to, one or more other therapeutic agents (e.g., desired known immunosuppressive therapeutics).

It will be appreciated that the therapies employed may achieve a desired effect for the same disorder (for example, a provided tissue component composition useful for treating transplant rejection may be administered concurrently with a known immunosuppressant therapeutic that is also useful for treating transplant rejection), or they may achieve different effects (for example, a provided tissue component composition that is useful for treating transplant rejection may be administered concurrently with a therapeutic agent that is useful for alleviating adverse side effects, for instance, inflammation, nausea, etc.). In some embodiments, provided tissue component compositions in accordance with the invention are administered with a second therapeutic agent that is approved by the U.S. Food and Drug Administration (FDA).

As used herein, the terms “in combination with” and “in conjunction with” mean that the provided nanoparticle formulations can be administered concurrently with, prior to, or subsequent to, one or more other desired therapeutics. In general, each substance will be administered at a dose and/or on a time schedule determined for that agent.

Dosing

In some embodiments, provided pharmaceutical formulations are administered according to a dosing regimen sufficient to achieve a desired immunological reaction. For example, in some embodiments, a dosing regimen is sufficient to achieve a desired immunological reaction if its administration to a relevant patient population shows a statistically significant correlation with achievement of the desired immunological reaction.

In some embodiments, the desired immunological reaction is a reduction in the degree and/or prevalence of symptoms of transplant rejection of at least about 20%, about 25%; about 30%; about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In some embodiments, a provided pharmaceutical formulation is administered according to a dosing regimen sufficient to achieve a reduction in the degree and/or prevalence of symptoms of transplant rejection of a specified percentage of a population of patients to which the formulation is administered. In some embodiments, the specified percentage of population of patients to which the formulation was administered is at least about 5%, about 10%, about 15%, about 20%, about 25%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

To give but a few illustrative examples, in some embodiments, administration of at least one provided pharmaceutical formulation according to a dosing regimen is sufficient to achieve a reduction in the degree and/or prevalence of transplant rejection of at least about 20% in at least about 50% of the population of patients to which the formulation was administered. In some embodiments, administration of at least one provided pharmaceutical formulation according to a dosing regimen is sufficient to achieve a reduction in the degree and/or prevalence of transplant rejection of at least about 30% in at least about 50% of the population of patients to which the formulation was administered.

In some embodiments, at least one provided pharmaceutical formulation is administered according to a dosing regimen sufficient to achieve a delay in the onset of symptoms of transplant rejection. In some embodiments, at least one provided pharmaceutical formulation is administered according to a dosing regimen sufficient to prevent the onset of one or more symptoms of transplant rejection.

In some embodiments, a provided dosing regimen comprises or consists of a single dose. In some embodiments, a provided dosing regimen comprises or consists of multiple doses, separated from one another by intervals of time that may or may not vary. In some embodiments, a provided dosing regimen comprises or consists of dosing once every 20 years, once every 10 years, once every 5 years, once every 4 years, once every 3 years, once every 2 years, once per year, twice per year, 3 times per year, 4 times per year, 5 times per year, 6 times per year, 7 times per year, 8 times per year, 9 times per year, 10 times per year, 11 times per year, once per month, twice per month, three times per month, once per week, twice per week, three times per week, 4 times per week, 5 times per week, 6 times per week, daily, twice daily, 3 times daily, 4 times daily, 5 times daily, 6 times daily, 7 times daily, 8 times daily, 9 times daily, 10 times daily, 11 times daily, 12 times daily, or hourly.

In some embodiments, a provided dosing regimen comprises or consists of an initial dose with one or more booster doses. In some embodiments, one or more booster doses are administered 1 day, 2 days, 3 days, 4 days, 5 days, 6 days, 1 week, 2 weeks, 1 month, 2 months, 6 months, 1 year, 2 years, 5 years, 10 years, or longer than 10 years after the initial dose. In some embodiments, an initial dose comprises a series of doses administered over a period of time. For example, in some embodiments, an initial dose comprises a series of 1, 2, 3, 4, 5, 6, 7, 8, 9, 10, 11, 12, 13, 14, 15, 16, 17, 18, 19, 20, 21, 22, 23, 24, or more doses administered at regular intervals, e.g., intervals that are close in time to one another, such as 5 minute intervals, 10 minute intervals, 15 minute intervals, 20 minute intervals, 25 minute intervals, 30 minute intervals, 45 minute intervals, hourly intervals, every 2 hours, etc.

In some embodiments, an initial dose and booster doses contain the same amount of provided tissue components and/or tissue component compositions. In some embodiments, an initial dose and booster doses contain different amounts of provided tissue component composition. In certain embodiments, provided tissue component compositions are administered at dosage levels sufficient to deliver from about 0.001 mg/kg to about 100 mg/kg, from about 0.01 mg/kg to about 50 mg/kg, preferably from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day. In some embodiments, provided tissue components compositions are administered at a dose equal to or greater than 0.001 mg/kg/day, 0.01 mg/kg/day, 0.05 mg/kg/day, 0.1 mg/kg/day, 0.5 mg/kg/day, 1 mg/kg/day, 5 mg/kg/day, 10 mg/kg/day, 50 mg/kg/day, 100 mg/kg/day, 500 mg/kg/day, or 1,000 mg/kg/day. In some embodiments, a daily dose is given in one administration. In some embodiments, a provided daily dose is given in two or more administration per day (e.g., 2, 3, 4, or 5).

In some embodiments, provided tissue component compositions are formulated into a unit dose. In some embodiments, a unit dosage is about 10 mg, about 25 mg, about 50 mg, about 100 mg, about 250 mg, about 500 mg, about 1 g, about 5 g, about 10 g, about 25 g, about 50 g, about 100 g, or more than about 100 g. In some embodiments, the amount of provided tissue component composition present in a particular unit dose depends on the subject to which the formulation is to be administered. To give but a few examples, in some embodiments, a unit dose appropriate for a mouse is smaller than a unit dose that is appropriate for a rat, which is smaller than a unit dose that is appropriate for a dog, is smaller than a unit dose that is appropriate for a human.

In some embodiments, a provided dosing regimen comprises or consists of administration of multiple doses over the course of the subject's entire lifespan. In some embodiments, a provided dosing regimen comprises administration of multiple doses over the course of several years (e.g., 2, 3, 4, 5, 6, 7, 8, 9, 10, 15, 20, 25, 30, 35, 40, 45, 50, 60, 70, 80, 90, or 100 years). In some embodiments, a provided dosing regimen comprises or consists of multiple doses over the course of 1, 2, 3, 4, 5, 6, 7, 8, 9, 10, 11, or 12 months.

The invention will be more fully understood by reference to the following examples. They should not, however, be construed as limiting the scope of the invention. All literature citations are incorporated by reference.

### EXEMPLIFICATION

### Example 1

### Patient-Specific Desensitization to Prevent Craft Rejection

The present Example describes the use of certain embodiments in patient-specific desensitization pre-transplant to prevent graft rejection.

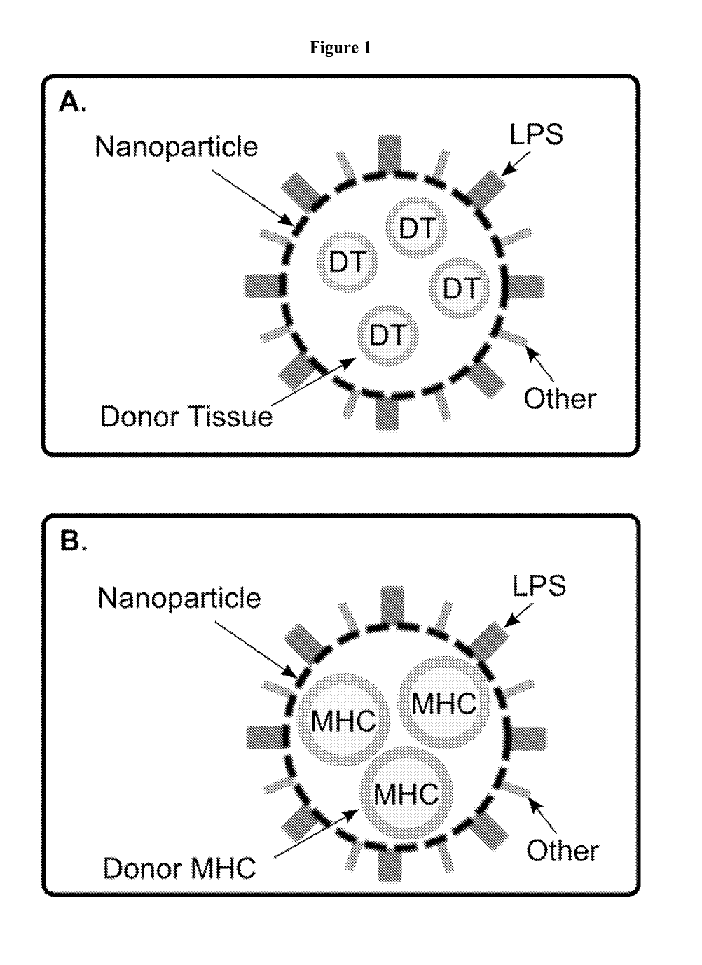

In this example, a variety of MHC protein is available in multiple drug forms (e.g., encapsulated in one or more nanoparticles, microbial cells, etc.). Specifically, each MHC protein is encapsulated within a distinct population of nanoparticles (e.g., bottles of nanoparticle compositions, each bottle containing nanoparticles encapsulating a specific MHC protein), for example, as shown in FIG. 1B. In some embodiments, each MHC protein is encapsulated within a microbial cell (e.g., bottles of dead E. coli, each bottle containing E. coli encapsulating a specific MHC protein). In this Example, a kit comprises a set of nanoparticles and/or microbial cells, each nanoparticle and/or microbial cell encapsulating an individual MHC (e.g., each drug in kit contains an individual MHC). In some embodiments, a kit may comprise mixtures of nanoparticles and/or microbial cells, wherein one or more nanoparticle and/or microbial cells, each encapsulating individual MHCs, are packaged together (e.g., a drug comprises two or more individual MHCs, i.e. two different nanoparticles and/or microbial cells).

As a first step, MHC mismatch between donor and recipient (i.e., patient) is determined. Briefly, the donor is tissue-typed to determine which MHC proteins are expressed by the donor. The recipient is tissue-typed to determine which MHC proteins are expressed by the recipient. The lists of donor and recipient MHC proteins are compared to identify those MHC proteins that are expressed by the donor and not expressed by the recipient.

A mixture of appropriate MHC proteins is prepared as described above (e.g., using one of various drug forms and/or kits described above including nanoparticles and/or microbial cells) and administered to the patient (i.e., recipient). The compositions are administered orally, though other routes of administration could also be used.

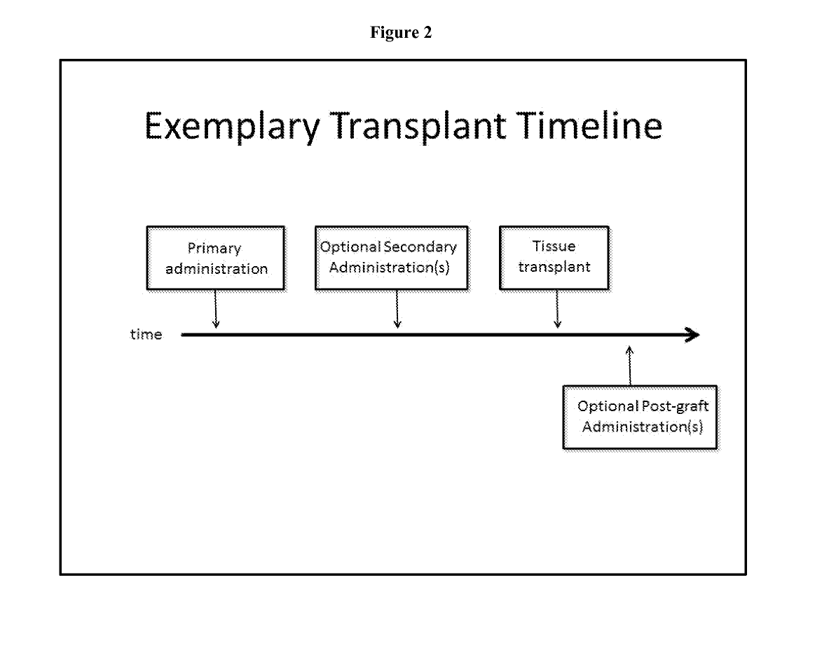

Administration of donor tissue components, including donor MHC proteins to a recipient before a transplant results in desensitization and tolerance of the recipient to donor MHC proteins, and increases the likelihood of a successful transplant with reduced risk of graft rejection post-transplant. An exemplary timeline showing administration of provided nanoparticles is shown in FIG. 2.

### Example 2

### Host Desensitization to Prevent Craft Rejection

The present Example describes host-specific desensitization post-transplant to prevent graft rejection, for use in accordance with the present invention.

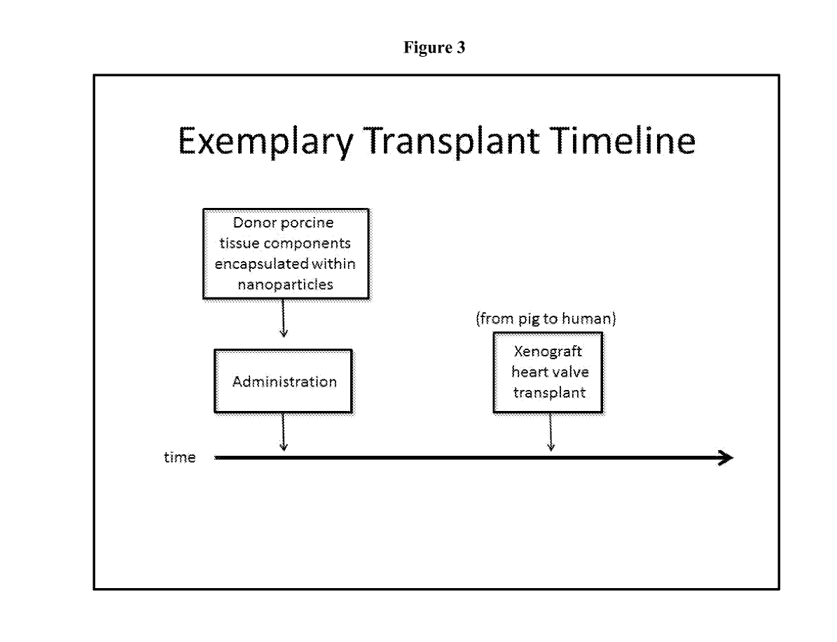

Donor tissue components are isolated and encapsulated within nanoparticles, for example, as shown in FIG. 1A. For this example, a xenograft heart valve transplant is being performed (e.g., from pig to human), and donor porcine tissue components are isolated and encapsulated within nanoparticles according to methods described above. An exemplary timeline is provided in FIG. 3. This procedure could easily applied in other situations, for example, in the case of a bone marrow transplant, donor bone tissue components are isolated and encapsulated within nanoparticles.

Nanoparticle compositions comprising encapsulated donor tissue components are administered to a host subsequent to a transplant in order to reduce, modulate, and/or eliminate host immune response to the transplanted tissue components.

### Example 3

### Donor Desensitization to Prevent Craft Rejection

The present Example describes donor-specific pre-transplant desensitization to prevent host graft rejection, for use in accordance with the present invention.

Recipient tissue components are isolated and encapsulated within nanoparticles. In this example, again a xenograft heart valve transplant (e.g., from pig to human), recipient tissue components are isolated and encapsulated within nanoparticles, and administered to the donor pig.

Nanoparticle compositions comprising encapsulated recipient tissue components are administered to a donor before a transplant to desensitize the donor to recipient MHC proteins. Administration of recipient tissue components to a donor before a transplant results in desensitization and tolerance of the donor to recipient MHC proteins, and increases the likelihood of a successful transplant with reduced risk of graft rejection post-transplant.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method comprising steps of
administering to a recipient organism who has received or will receive a transplant of one or more heterologous tissue components from a donor organism a composition comprising encapsulated donor organism tissue components.

2. The method of claim 1, wherein the one or more heterologous tissue components are encapsulated within a nanoparticle.

3. The method of claim 1, wherein the one or more heterologous tissue components are encapsulated within a microbial cell.

4. The method of claim 3, wherein the microbial cell is or comprises a bacterial, fungal, archaeal or protozoan cell.

5. The method of claim 3 or 4, wherein the microbial cell is or comprises an E. coli cell.

6. The method of any of claims 1-5, wherein the composition is administered prior to the transplant.

7. The method of any of claims 1-5, wherein the composition is administered subsequent to the transplant.

8. The method of any one of the preceding claims, wherein the composition is administered intravenously, intradermally, transdermally, orally, subcutaneously, and/or transmucosally.

9. The method of claim 8, wherein transmucosal administration is buccal, nasal, bronchial, vaginal, rectal, and/or sublingual administration.

10. A method comprising
administering to a donor organism from which one or more donor tissue components are to be transplanted into a recipient organism a composition comprising one or more encapsulated recipient tissue components.

11. The method of claim 10, wherein the one or more encapsulated recipient tissue components are encapsulated within a nanoparticle.

12. The method of claim 10, wherein the one or more one or more encapsulated recipient tissue components are encapsulated within a microbial cell.

13. The method of claim 12, wherein the microbial cell is a bacterial, fungal, archaeal or protozoan cell.

14. The method of claim 12 or 13, wherein the microbial cell is an E. coli cell.

15. The method of any of claims 10-14, wherein the composition is administered prior to a transplant.

16. The method of any of claims 10-14, wherein the composition is administered subsequent to a transplant.

17. The method of any one of the preceding claims, wherein the composition is administered intravenously, intradermally, transdermally, orally, subcutaneously, and/or transmucosally.

18. The method of claim 17, wherein transmucosal administration is buccal, nasal, bronchial, vaginal, rectal, and/or sublingual administration.

19. A method comprising
determining which MHC proteins are expressed by a donor organism;
determining which MHC proteins are expressed by a recipient organism;
selecting one or more encapsulated MHC proteins that matches one or more MHC proteins of the donor organism; and
administering to the recipient organism into which one or more donor tissue components are to be transplanted from the donor organism the one or more encapsulated MHC proteins.

20. The method of claim 19, wherein the one or more encapsulated MHC proteins are encapsulated within a nanoparticle.

21. The method of claim 19, wherein the one or more encapsulated MHC proteins are encapsulated within a microbial cell.

22. The method of claim 21, wherein the microbial cell is a bacterial, fungal, archael or protozoan cell.

23. The method of claim 21 or 22, wherein the microbial cell is an e. coli cell.

24. The method of any of claims 19-23, wherein the composition is administered prior to a transplant.

25. The method of any of claims 19-23, wherein the composition is administered subsequent to a transplant.

26. The method of any one of the preceding claims, wherein the composition is administered intravenously, intradermally, transdermally, orally, subcutaneously, and/or transmucosally.

27. The method of claim 26, wherein transmucosal administration is buccal, nasal, bronchial, vaginal, rectal, and/or sublingual administration.
